SEQUENCE LISTING (44 sequences)

SEQ ID NO: 1 - protein, 17 aa, Artificial Sequence
  Synthetic Peptide
  misc_feature (17)..(17): Xaa can be any naturally occurring amino acid
GFRQNQKEGRCSHSLNX

SEQ ID NO: 2 - DNA, 133 bp, Artificial Sequence
  Synthetic Primer
atctatatagctattttttttttaacttcctttattttccttacagggttttagacaaaatcaaaaagaaggaaggtgctcacattccttaaattaaggagtaagtctgccagcattatgaaagtgaatctta

SEQ ID NO: 3 - DNA, 108 bp, Artificial Sequence
  Synthetic Primer
ttttaacttcctttattttccttacagagacaaaatcaaaaagaaggaaggtgctcacattccttaaattaaggagtaagtctgccagcattatgaaagtgaatctta

SEQ ID NO: 4 - DNA, 81 bp, Artificial Sequence
  Synthetic Primer
agacaaaatcaaaaagaaggaaggtgctcacattccttaaattaaggagtaagtctgccagcattatgaaagtgaatctta

SEQ ID NO: 5 - DNA, 81 bp, Artificial Sequence
  Synthetic Primer
agacaaaatcaaaaagaaggaaggtgctcacattccttaaattaaggagtaagtctgccagcattatgaacgtgaatctta

SEQ ID NO: 6 - DNA, 81 bp, Artificial Sequence
  Synthetic Primer
agacaaaatcaaaaagaaggaaggtgctcacattccttaaattaaggagtaagtatgccagcattatgaaagtgaatctta

SEQ ID NO: 7 - DNA, 80 bp, Artificial Sequence
  Synthetic Primer
gacaaaatcaaaaagaaggaaggtgctcacattccttaaattaaggagtaagtctgccagcattatgaaagtgaatctta

SEQ ID NO: 8 - DNA, 68 bp, Artificial Sequence
  Synthetic Primer
gacaaaatcaaaaagaaggaaggtgctcacattccttaaattaaggagtaagatgaaagtgaatctta

SEQ ID NO: 9 - DNA, 76 bp, Artificial Sequence
  Synthetic Primer
gacaaaatcaaaaagaaggaaggtgctcacattccttaaattaaggagtaagtctgccagcattatgaaagtctta

SEQ ID NO: 10 - DNA, 79 bp, Artificial Sequence
  Synthetic Primer
gacaaaatcaaaaagaaggaaggtgctcacattccttaaattaggagtaagtctgccagcattatgaaagtgaatctta

SEQ ID NO: 11 - DNA, 164 bp, Artificial Sequence
  Synthetic Primer
cagggtttcagacaaaatcaaaaagaaggaaggtgctcacattccttaaattaaggagtaagtctgccagcattatgaaagtgaatcttacttttgtaaaactttatggtttgtggaaaacaaatgtttttgaacatttaaaaagttcagatgttaaaaagttg

SEQ ID NO: 12 - DNA, 164 bp, Artificial Sequence
  Synthetic Primer
cagggttttagacaaaatcaaaaagaaggaaggtgctcacattccttaaattaaggagtaagtctgccagcattatgaaagtgaatcttacttttgtaaaactttatggtttgtggaaaacaaatgtttttgaacatttaaaaagttcagatgttagaaagttg

SEQ ID NO: 13 - DNA, 71 bp, Artificial Sequence
  Synthetic Primer
cctttattttccttacagggtttcagacaaaatcaaaaaggaaggaaggtgctcacattccttaaattaag

SEQ ID NO: 14 - DNA, 70 bp, Artificial Sequence
  Synthetic Primer
cctttattttccttacagggtttcagacaaaatcaaaaaggaaggaaggtgctcacattccttaattaag

SEQ ID NO: 15 - DNA, 70 bp, Artificial Sequence
  Synthetic Primer
cctttattttccttacagggtttcagacaaaatcaaaaggaaggaaggtgctcacattccttaaattaag

SEQ ID NO: 16 - DNA, 70 bp, Artificial Sequence
  Synthetic Primer
cctttatttccttacagggtttcagacaaaatcaaaaaggaaggaaggtgctcacattccttaaattaag

SEQ ID NO: 17 - DNA, 70 bp, Artificial Sequence; identical to SEQ ID NO: 15
  Synthetic Primer

SEQ ID NO: 18 - DNA, 71 bp, Artificial Sequence; identical to SEQ ID NO: 13
  Synthetic Primer

SEQ ID NO: 19 - DNA, 70 bp, Artificial Sequence
  Synthetic Primer
cctttattttccttacagggtttcagacaaaatcaaaaaggaaggaaggtctcacattccttaaattaag

SEQ ID NO: 20 - DNA, 70 bp, Artificial Sequence
  Synthetic Primer
cctttattttccttacagggtttcagacaaaatcaaaaaggaaggaaggtgccacattccttaaattaag

SEQ ID NO: 21 - DNA, 70 bp, Artificial Sequence
  Synthetic Primer
cctttattttccttacagggtttcagacaaaatcaaaaaggaaggaaggtgctcacattccttaaataag

SEQ ID NO: 22 - DNA, 70 bp, Artificial Sequence
  Synthetic Primer
cctttattttcctacagggtttcagacaaaatcaaaaaggaaggaaggtgctcacattccttaaattaag

SEQ ID NO: 23 - DNA, 71 bp, Artificial Sequence; identical to SEQ ID NO: 13
  Synthetic Primer

SEQ ID NO: 24 - DNA, 70 bp, Artificial Sequence
  Synthetic Primer
cctttattttccttacagggttttagacaaaatcaaaaagaaggaaggtgctcacattccttaaattaag

SEQ ID NO: 25 - DNA, 55 bp, Artificial Sequence
  Synthetic Primer
cctttatttagacaaaatcaaaaagaaggaaggtgctcacattccttaaattaag

SEQ ID NO: 26 - DNA, 57 bp, Artificial Sequence
  Synthetic Primer
cctttattttcagacaaaatcaaaaagaaggaaggtgctcacattccttaaattaag

SEQ ID NO: 27 - DNA, 68 bp, Artificial Sequence
  Synthetic Primer
cctttattttccttacagggttagacaaaatcaaaaagaaggaaggtgctcacattccttaaattaag

SEQ ID NO: 28 - DNA, 64 bp, Artificial Sequence
  Synthetic Primer
cctttattttccttacagagacaaaatcaaaaagaaggaaggtgctcacattccttaaattaag

SEQ ID NO: 29 - DNA, 46 bp, Artificial Sequence
  Synthetic Primer
agacaaaatcaaaaagaaggaaggtgctcacattccttaaattaag

SEQ ID NO: 30 - DNA, 45 bp, Artificial Sequence
  Synthetic Primer
gacaaaatcaaaaagaaggaaggtgctcacattccttaaattaag

SEQ ID NO: 31 - DNA, 106 bp, Artificial Sequence
  Synthetic Primer
ctatgtctatatagctattttttttaacttcctttattttccttacagggtttcagacaaaatcaaaaagaaggaaggtgctcacattccttaaattaaggagtaa

SEQ ID NO: 32 - DNA, 106 bp, Artificial Sequence; identical to SEQ ID NO: 31
  Synthetic Primer

SEQ ID NO: 33 - DNA, 106 bp, Artificial Sequence; identical to SEQ ID NO: 31
  Synthetic Primer

SEQ ID NO: 34 - DNA, 106 bp, Artificial Sequence; identical to SEQ ID NO: 31
  Synthetic Primer

SEQ ID NO: 35 - DNA, 32 bp, Artificial Sequence
  Synthetic Primer
atagctattttttttaacttcctttattttcc

SEQ ID NO: 36 - DNA, 22 bp, Artificial Sequence
  Synthetic Primer
tgagcaccttccttctttttga

SEQ ID NO: 37 - DNA, 15 bp, Artificial Sequence
  Synthetic Probe
  misc_feature (1)..(1): FAM tagged
ttgtctgaaaccctg

SEQ ID NO: 38 - DNA, 15 bp, Artificial Sequence
  Synthetic Probe
  misc_feature (1)..(1): VIC tagged
ttttgtctaaaaccc

SEQ ID NO: 39 - DNA, 26 bp, Artificial Sequence
  Synthetic Primer
gtgtggattaagatgactcttggtac

SEQ ID NO: 40 - DNA, 27 bp, Artificial Sequence
  Synthetic Primer
caccttccttctttttgattttgtctg

SEQ ID NO: 41 - DNA, 27 bp, Artificial Sequence
  Synthetic Primer
cttcctttattttccttacagggttcc

SEQ ID NO: 42 - DNA, 22 bp, Artificial Sequence
  Synthetic Primer
tacaatgaacagccatgtccac

SEQ ID NO: 43 - DNA, 45 bp, Artificial Sequence
  Synthetic Primer
tgtaaaacgacggccagtcttcctttattttccttacagggttgc

SEQ ID NO: 44 - DNA, 41 bp, Artificial Sequence
  Synthetic Primer
caggaaacagctatgaccaagtctgctggtctgcctactag

(19) United States
(12) Patent Application Publication
(10) Pub. No.: US 2019/0066842 A1
(43) Pub. Date: Feb. 28, 2019

(54) A NOVEL ALGORITHM FOR SMN1 AND SMN2 COPY NUMBER ANALYSIS USING COVERAGE DEPTH DATA FROM NEXT GENERATION SEQUENCING

(71) Applicants: BAYLOR COLLEGE OF MEDICINE, Houston, TX (US); BAYLOR MIRACA GENETICS LABORATORIES, LLC, Houston, TX (US)

(72) Inventors: Jinglan Zhang, Houston, TX (US); Lee-Jun C. Wong, Sugar Land, TX (US); Yanming Feng, Houston, TX (US); Xiaoyan Ge, Houston, TX (US)

(21) Appl. No.: 16/083,452

(22) PCT Filed: Mar. 9, 2017
(86) PCT No.: PCT/US17/21603

§ 371 Date: Sep. 7, 2018

Related U.S. Application Data
(60) Provisional application No. 62/305,780, filed on Mar. 9, 2016.

(51) Int. Cl. G16H 50/20; G06F 19/22; G06F 19/28

(52) U.S. Cl. CPC G16H 50/20; G06F 19/28; G06F 19/22

(57) ABSTRACT

The disclosure concerns methods and compositions for obtaining reliable copy numbers of highly homologous gene(s) using next generation sequencing. The methods determine whether or not an individual is a carrier of an autosomal recessive gene mutation using a determination of copy number of two genes, in specific embodiments. In at least some cases, an individual is identified whether or not he or she is a carrier or affected for a genetic defect in SMN1, wherein the defect is associated with spinal muscular atrophy.

This application claims priority to U.S. Provisional Patent Application Ser. No. 62/305,780, filed Mar. 9, 2016, which is incorporated by reference herein in its entirety.

### TECHNICAL FIELD

Embodiments of the disclosure concern at least the fields of genetics, cell biology, molecular biology, diagnostics, and medicine.

### BACKGROUND

Spinal muscular atrophy (SMA, MIM #253300) is a neuromuscular disorder caused by the loss of motor neurons in the spinal cord and the brainstem leading to generalized muscle weakness and muscular atrophy which impair activities such as crawling, walking, sitting up, and controlling head movement (Emery, et al., 1976). SMA has a variable expressivity with a broad range of onset and severity. In severe cases, death occurs within the first two years of life mostly due to respiratory failure (Dubowitz, 1995). SMA is the second most common autosomal recessive disorder after cystic fibrosis (CF), with an incidence of about 1 in 10,000 live births and a carrier frequency of about 1/40 to 1/100 in different ethnic groups, with lower carrier frequencies in African Americans and Hispanics (Swoboda, et al., 2005; Hendrickson, et al., 2009; Prior, et al., 2008; MacDonald, et al., 2014). SMA is caused by mutations in the survival motor neuron 1 (SMN1) gene including deletions, gene conversions or intragenic mutations in both of the SMN1 alleles, while SMN2 copy number may modify the disease severity (Feldkotter, et al., 2002). SMN1 and SMN2 are highly homologous, and only differ by five base pairs, none of which change the amino acid sequences. A single C to T change in SMN2 exon 7 (c.840C>T) affects an exonic splicing enhancer (ESE) or creates an exon silencer element (ESS) that results in the majority of transcripts lacking exon 7 (Cartegni et al., 2002; Kashima and Manley, 2003), which results in a reduction of full-length transcripts from SMN2 (Lorson, et al., 1999).

SMA has unique features that can be recognized clinically that often prompt follow-up molecular diagnosis. RFLP is commonly used as a diagnostic test for SMA patients, while it cannot detect carrier status. The first carrier test for SMA was developed in 1997 using a competitive PCR strategy for the quantitative analysis of SMN1 copy numbers which set the foundation for carrier screening for SMA (McAndrew, et al., 1997). With the advancement of technology in the last two decades, high-throughput methods were developed using MLPA or quantitative PCR which enabled expanded population SMA carrier screening most of which involve SMN1 copy numbers. Although the whole gene or exonic copy number variations (CNVs) account for the majority of SMA disease alleles, ˜2.5% of SMA pathogenic variants are point mutations (MacDonald, et al., 2014). Apparently, carriers of such small pathogenic variants would be missed by current mainstay carrier testing methods which focus on interrogating the c.840C>T locus with or without other gene specific loci. In addition, silent carriers who have two copies of SMN1 (duplication allele) on one chromosome 5 and zero on the other (2+0) are beyond the scope of SMN1 copy number analysis for carrier tests. To reduce the false negative rate in carrier testing, sequence variant polymorphisms tightly linked to the SMN1 duplication allele were used as markers for SMA silent carrier detection in some populations (Luo, et al., 2014).

The clinical application of NGS technologies has rapidly transformed medicine as a cost effective approach to search pathogenic variants in patients affected with genetic disease on a genome scale (Yang, et al., 2014). NGS-based carrier screening panel has also been developed which offers greater clinical outcomes with increased detection rate and lower total healthcare cost compared to conventional genotyping or other targeted approach (Hallam, et al., 2014). The comprehensiveness of NGS testing makes receiving a negative result much more reassuring in terms of residual risk of sequence variants detected. Importantly, NGS has been shown by us and others that it can discover CNVs at both gene and exonic levels for clinical tests (Feng, et al., 2015; Retterer, et al., 2015). The capability to detect such pathogenic variants when performing carrier screening by NGS is particularly important for diseases with high percentage of pathogenic variants caused by CNVs. However, NGS based CNV detection in general is still challenging for small deletions/duplications at single exon or sub-exon level due to technical noises introduced by uneven coverage in regions with different GC contents, non-linear amplification by PCR, or inter-run variations caused by other assay artifacts known as batch effects. Another drawback for CNV analysis by NGS is the lack of locus-specific computational program for genes with homologous sequences requiring accurate alignment of gene specific reads and subsequent copy number analysis. Therefore, such genes including SMN1/2 are normally not included in NGS secondary analysis for variant calling, or variant calling in these genes often fail mapping quality filter.

The present disclosure satisfies a long felt need in the art to employ NGS for highly homologous sequences, at least to determine their gene copy number, and also provides a long felt need in the art for reliable testing for carrier status for SMA.

### BRIEF SUMMARY

Embodiments of the disclosure concern methods and compositions for analysis of one or more samples from an individual. In specific embodiments, the disclosure concerns determination of whether or not an individual has an allele that includes at least one specific gene sequence and/or polymorphism and/or mutation and/or copy number. Thus, in some cases DNA from a sample from an individual is analyzed to determine if the individual has certain copy number(s) of one or more genes that would classify the individual as a carrier for a disease. In at least some cases, a pair of genes in question is one in which the genes are nearly identical (for example, greater than 95, 96, 97, 98, 99, 99.1, 99.2, 99.3, 99.4, 99.5, 99.6, 99.7, 99.8, or 99.9% identity) or otherwise has significant sequence similarity to another gene, such as the pair being a gene and a pseudogene or paralogue gene, for example (such as SMN1/SMN2, CYP21A2/CYP21A1P, or HBA1/HBA2). The pair of genes that are in need of determination of copy number may have a difference of only 1, 2, 3, 4, 5, or more nucleotides.

The methods allow one to utilize sequencing data from NGS to determine copy number of one or more genes. Embodiments of the disclosure utilize counts of single instances of a particular sequenced region (every single sequenced DNA fragment may be referred to as one “read”) that corresponds to all or part of exons for a certain gene. The counts, therefore, are a representative and corresponding value of the copy number of a region of a gene and, thereby, of the gene itself. In some aspects to the methods, the reads that comprise sequence that does not encompass one or more signature variants (such as single nucleotide polymorphisms (SNPs) or single nucleotide variants (SNVs)) between a first and second gene are utilized for determination of total copy number of both of a first and second gene but are not utilized for determination of copy number ratio between a first and second gene. In other aspects to the methods, the reads that comprise sequence that does encompass one or more signature variants are utilized for determination of a ratio of copy number between a first and second gene but are not utilized for determination of total copy number of both of a first and second gene. That is, in specific embodiments of the methods there is no distinguishing between the two genes when the determining of the total copy number value for the ultimate computation.

The disclosure encompasses methods for determining whether or not an individual is a carrier for a genotype associated with SMA, including in at least some cases determining the severity of the affliction with SMA. At least some methods described herein analyze copy number of both SMN1 and SMN2. Certain methods allow for the use of next generation sequencing (NGS) using analysis of SMN1 and SMN2 even though they are highly similar in sequence identity. The methods exploit the minimal differences between the two genes. Methods described herein for genetic analysis may be used as a sole test for an individual or may be employed as one of multiple tests for an individual.

Some methods of the disclosure determine whether or not an individual is a carrier for SMA. In particular embodiments, the DNA of an individual is analyzed for copy number of SMN1 and SMN2. The ratio and/or total copy number of one or more genes, including SMN1 and SMN2, are encompassed as part of analyses herein. The analysis of an individual's DNA using methods of the disclosure can allow for determination whether or not an individual is a carrier for spinal muscular atrophy (SMA), for example. In particular embodiments, methods and compositions for distinguishing SMN1 and/or SMN2 copy number(s) utilize as part of the method the determination of a variance between SMN1 and SMN2 at a particular exon or intron, such as exons 7 and 8 or introns 6 and 7.

Compositions for carrier screen tests are encompassed in the disclosure. The carrier screen tests may be utilized with other types of tests, including other carrier screen tests, or the composition may solely be utilized for determination of carrier status for a particular genetic mutation and related disease.

In some embodiments, there is provided a method of determining gene copy number for an individual, comprising the step of identifying copy number of two nearly identical genes using sequencing data from next generation sequencing to distinguish at least one variance between the two genes. In specific embodiments, the identifying step comprises the determination of a mathematical relationship between a) the copy number ratio of the two genes, and b) the total copy number for both of the two genes in sum. In certain embodiments, the mathematical relationship is further defined as computing copy number for each gene by applying the copy number ratio to the total copy number. In certain cases, the two genes are SMN1 and SMN2. In at least some cases, the gene copy number identifies carrier status for an individual, and the gene copy number may be 0, 1, 2, 3, 4, 5, 6, 7, or more.

In certain embodiments, there is provided a method of assaying nucleic acid from a sample from an individual for a recessive allele for a genetic mutation associated with spinal muscular atrophy (SMA), comprising the step of generating a mathematical relationship between the total copy number of SMN1 and SMN2 and the copy number ratio of SMN1 to SMN2, wherein the total copy number and copy number ratio are determined using next generation sequencing data. The method may further comprise the step of determining that an individual is in need of assaying for the allele. In certain cases, the individual has a family history of SMA. The individual may be pregnant. The individual may be in need of family planning.

In particular embodiments, there is provided a method, comprising: receiving sequenced sample data; determining a copy number ratio between two nearly identical genes of the received sample data; determining a total copy number of the two nearly identical genes of the received sample data; and determining a final copy number for the two nearly identical genes for the received sample. In specific embodiments, the method further comprises determining a patient outcome hypothesis based, at least in part, on the determined final copy number for the received sample corresponding to the patient. In some cases, the step of determining the patient outcome hypothesis comprises determining that a patient is a carrier when the final copy number is not equal to two. The received sequenced sample data may be received from next generation sequencing (NGS) and the sample data may be aligned to hg19, for example. In specific embodiments, the received sequenced sample data comprise a plurality of samples corresponding to a plurality of patients, and wherein a copy number ratio, a total copy number, and a final copy number is determined for each of the plurality of samples. The two nearly identical genes may comprise the SMN1 and SMN2 genes. The step of determining the copy number ratio may comprise reading a depth(rd) of PSVs for the received sample data; calculating a copy number ratio for the received sample data for predetermined exons selected based on exons with expected differences; and building a table of calculations for the calculated copy number ratios for a plurality of samples. In certain cases, the step of determining the total copy number may comprise determining a total coverage of selected exons of the two nearly identical genes for each of a plurality of received samples; determining a median or mean of each of the selected exons from samples having a ratio of the two nearly identical genes equal to approximately one; normalizing the total coverage for the selected exons for each sample of the plurality of samples relative to all samples of the plurality of samples; and determining the total copy number for each of the selected exons for each of the plurality of samples based, at least in part, on the normalized total coverage.

In some embodiments, there is an apparatus comprising a processor and a memory, wherein the processor is coupled to the memory, and wherein the processor is configured to perform the steps recited in any of methods encompassed by the disclosure.

In certain embodiments, there is a computer program product, comprising: a non-transitory computer readable medium comprising code to perform steps comprising the steps recited in any of the methods encompassed by the disclosure.

The foregoing has outlined rather broadly the features and technical advantages of the present disclosure in order that the detailed description of the disclosure that follows may be better understood. Additional features and advantages of the disclosure will be described hereinafter which form the subject of the claims of the disclosure. It should be appreciated by those skilled in the art that the conception and specific embodiment disclosed may be readily utilized as a basis for modifying or designing other structures for carrying out the same purposes of the present disclosure. It should also be realized by those skilled in the art that such equivalent constructions do not depart from the spirit and scope of the disclosure as set forth in the appended claims. The novel features which are believed to be characteristic of the disclosure, both as to its organization and method of operation, together with further objects and advantages will be better understood from the following description when considered in connection with the accompanying figures. It is to be expressly understood, however, that each of the figures is provided for the purpose of illustration and description only and is not intended as a definition of the limits of the present disclosure.

### BRIEF DESCRIPTION OF THE DRAWINGS

FIGS. 1A-F show an example of NGS data processing for SMN1 and SMN2 copy number analysis.

FIG. 2 demonstrates a SMN1:SMN2 copy number ratio distribution in 2,488 pan-ethnic group individuals.

FIG. 3 shows the SMN1 and SMN2 copy number(s) distribution in 2,488 pan-ethnic group individuals.

FIG. 4 shows a sample with two copies of SMN1 and zero copy of SMN2 in which all reads that mapped to E7 and E8 of SMN2 were those without SMN1 PSVs (SEQ ID NOS 1-10).

FIGS. 5A-5D shows a representative batch of capture NGS data for SMN1 copy number detection.

FIG. 6 illustrates general embodiments of at least some steps of the methods that include alignment of pair-end reads (reads anchored by single gene-specific variants) to SMN1 or SMN2 locus.

FIG. 7 is a schematic block diagram illustrating one embodiment of a system for multi-attribute clustering.

FIG. 8 is a schematic block diagram illustrating one embodiment of a database system for multi-attribute clustering.

FIG. 9 is a schematic block diagram illustrating one embodiment of a computer system that may be used in accordance with certain embodiments of the system for multi-attribute clustering.

FIGS. 10A-10C is the SMN1 and SMN2 NGS sequence alignment surrounding the functional PSV at c.840. (FIG. 10A) The SI MN gene PSV1 (c.840C/T), PSV2 (c.888+100A/G) and SMN1 SNP g.27134T>G are located within a 148 bp region spanning exon 7 and intron 7 of the SMN1 or SMN2 gene, (FIG. 10B) The alignment of pair-end sequence reads (2×100) in a normal and SMN1/SMN2 gene hybrid sample. The red or purple box represents the pair-end read R1 or R2 respectively. The green letters at the PSV1, PSV2 or the SMN1 SNP loci indicate that the aligned reads match the reference sequence at these positions. Yellow letters indicate the mismatched bases in the correctly aligned reads due to sequence polymorphism or a gene conversion event. Red letters indicate the mismatched bases in the misaligned reads caused by sequence polymorphism or gene conversion. (FIG. 10(C) Sequence pileups of read pairs at the correct SMN1 locus (top) (SEQ ID NO: 11) and incorrect SMN2 locus (bottom) (SEQ ID NO: 12) (SEQ ID NO: 1).

FIG. 11 is a novel computational algorithm PGCNARS (paralogous gene copy number analysis by ratio and sum) for SMN1 copy number analysis using NGS coverage depth data for SMA carrier screening. PGCNARS involves three major steps for the SMN1 copy number analysis. Firstly, for each sample in the same capture pool, the copy number ratio of SMN1 to SMN2 is calculated using the read-depth of the PSVs in the exon 7 (c.840C/T) or exon 8 (c.*233T/A) of SMN1 and SMN2 (step a1-3). The SMN1 and SMN2 total copy number was determined by their exonic coverage data after normalization to the read depth of the median identified in the sample group (step b1-7). Lastly, the SMN1 copy number in each sample is calculated based on the SMN1 to SMN2 copy number ratio and their total copy number (step c).

FIGS. 12A-12B is a paralogous sequence variant (PSV) can be informative for NGS read alignment for highly homologous genes. The pileup for NGS reads for a sample with two copies of SMN1 (SEQ ID NOS:13-23) and zero copy of SMN2 (SEQ ID NOS:24-30) was shown surrounding the functional PSV c.840 (SEQ ID NO: 1). All reads mapped to SMN1 were those with the functional PSV (FIG. 12A) while the misaligned reads to SMN2 lack the PSV (FIG. 12B).

FIG. 13 is SMN1 and SMN2 alignment and copy number analysis were confounded by gene hybrids and SNP. A group of eight samples with three copies of SMN1, one copy of SMN2 and an SMN1 SNP (g.27134T>G) were aligned using pair-end (PE) and single-end (SE) mapping algorithm. The SMN1 and SMN2 copy number analyses were performed using the coverage data generated by the PE or SE alignment algorithm. The PE method underestimated SMN1 to SMN2 copy number ratio (left panel) and SMN1 copy number (middle panel) and the SMN2 copy number was overestimated (right panel).

FIGS. 14A-14C is distribution of SMN1 to SMN2 copy number ratios and SMN1 and SMN2 copy numbers in 6,738 samples. (FIG. 14A) There are four major groups of samples with different SMN1 to SMN2 copy number ratios approximately at 1, 2, 3, and ∞ (zero copy of SMN2). (FIG. 14B) The relative distributions of samples with different SMN1 copy numbers in 6,738 samples. (FIG. 14C) The relative distributions of samples with different SMN2 copy numbers in 6,738 samples.

FIG. 15 is a pedigree of a representative SMA family analyzed by NGS. Pedigree and the NGS pileup showed two children affected by SMA with zero copy SMN1. Both parents were carriers with one copy of SMN1 (SEQ ID NOS:31-34).

FIG. 16 is gene specific PCR was used to amplify the SMN1 gene to confirm sequence variants identified by capture NGS. Two fragments (5′ and 3′ fragment) were amplified using a gene specific primer designed based on exon 7 PSV and non-specific primers upstream (exon 2 primer) and downstream (exon 8 primer) of the PSV. Controls used in this study included DNA with two copies of SMN1, zero copy of SMN1 (SMA) and zero copy of SMN2.

FIG. 17 is RFLP analysis specifically detected the g.27134T>G SNP in the SMN1 locus. PCR was performed to amplify the SMN1 fragment containing the 2+0 carrier SNP (g.27134T>G). Primers were designed to specifically amplify SMN1, but not SMN2, by utilizing the c.840C PSV at exon 7, as well as an additional mismatch base pair before the PSV. HpyCH4III cut SMN1 PCR product only when SNP g.27134T>G was present. Controls were included (from left to right): DNA with a heterozygous SNP g.27134T>G in SMN1 producing digested PCR products of 173 bp, 235 bp and 408 bp in size, DNA without the g.27134T>G SNP, DNA with a homozygous g.27134T>G SNP, DNA with zero copy of SMN1 copy and no template control (NTC).

FIG. 18 is a haplotype with misaligned g.27134T>G SNP. (a) An SMN1 allele positive for the g.27134T>G SNP. (b) An SMN1 allele positive for the g.27134T>G SNP with the intron 7 PSV1 G converted to A. In this situation, the g.27134T>G SNP was misaligned to the SMN2 locus by NGS, but SMN1 specific RFLP analysis was able to correctly identify it in the SMN1 locus.

### DETAILED DESCRIPTION

As used herein the specification, “a” or “an” may mean one or more. As used herein in the claim(s), when used in conjunction with the word “comprising”, the words “a” or “an” may mean one or more than one. As used herein “another” may mean at least a second or more. In specific embodiments, aspects of the invention may “consist essentially of” or “consist of” one or more sequences of the invention, for example. Some embodiments of the invention may consist of or consist essentially of one or more elements, method steps, and/or methods of the invention. It is contemplated that any method or composition described herein can be implemented with respect to any other method or composition described herein.

The scope of the present application is not intended to be limited to the particular embodiments of the process, machine, manufacture, composition of matter, means, methods and steps described in the specification.

Embodiments of the disclosure allow determination of gene copy number using NGS data for genes that have highly homologous regions. Methods of the disclosure may be employed following next generation sequencing or third generation sequencing for determining copy number of two highly homologous genes or of determining copy number for a gene and a pseudogene. The determination of copy number in such situations may be informative for a medical purpose, such as determining whether or not an individual is a carrier, affected, or at risk for particular genetic disease(s).

Embodiments of the disclosure concern clinical molecular testing including carrier screening using NGS for testing for a particular carrier status for a disease in an individual.

The present disclosure concerns methods for analyzing copy number of SMN1 and SMN2 (as examples) for screening for whether or not a particular individual is a carrier for SMA, for example. The methods employ next generation sequencing including gene-specific reads by utilizing fragments having unique nucleotide(s) for SMN1 and/or SMN2. The methods of the disclosure avoid the use of primers or probes that target particular single nucleotide polymorphisms (SNPs). Embodiments of the disclosure are useful for determining copy number using NGS methods including for those genes with homologous sequences necessitating accurate alignment of gene specific reads and subsequent copy number analysis. Methods of the disclosure allow for enhanced variant calling using NGS in gene(s) that are difficult to analyze with NGS, particularly when the analysis requires or would benefit from reliable copy number analysis.

As an aspect to methods of the disclosure, determination of a copy number ratio between a first gene and a second gene that are highly identical to each other in sequence utilizes one or more informative variants (such as polymorphisms or mutations) that allow accurate alignment of multiple reads over a particular exon present in both genes, and this alignment facilitates accurate quantitation of the reads.

Methods of the disclosure utilize read depths of gene specific reads to calculate copy number ratio of a first gene to a second gene. In at least some cases, non-discriminating reads are utilized to calculate total copy number using all exons.

As an example, embodiments of this disclosure allow for Next Generation Sequencing or Third Generation Sequencing coverage data to call SMN1/SMN2 copy numbers. The highly homologous gene SMN2 makes the short NGS reads difficult to be aligned to the gene specific locus of SMN1 or SMN2. In addition, NGS is semi-quantitative in that the copy number analysis by NGS data is impacted by a lot of variables in library preparation, PCR cycle numbers, and sequencing artifacts. To overcome these problems, the inventors deployed a method decoupled the pair-end reads and performed alignment based on single-end reads to increase mapping specificity (reads anchored to gene specific locus by gene specific variants) to SMN1 or SMN2 locus. Gene-specific reads were counted by surveying fragments with at least one of the SMN1/2 unique nucleotides in order to calculate SMN1:SMN2 copy number ratios. Total SMN1 and SMN2 copy numbers were independently determined by counting all of the exon 7 and neighboring exons' reads. Together with SMN1 and SMN2 total copy and their copy number ratio, SMN1 and SMN2 gene copy numbers were determined.

In particular embodiments, a first step in the methods includes alignment of reads according to one or more nucleotides that differentiate between a first gene and a second gene. In a next step, one can calculate a copy number ratio of how many reads are aligned for the first gene versus how many reads are aligned for a second gene. Following this, a total copy number as a sum of both genes is determined. The value of the total copy number and the value of the copy number ratio allow interpretation of the exact copy number of the first and second genes. For example, if the total copy number for a particular sample is calculated to be 3 and the copy number ratio of 1:2 is determined based on the number of aligned reads according to a single differentiating nucleotide, then the actual copy number of the first gene is 1 and the actual copy number of the second gene is 2.

In some cases, a signature variance between two genes for use in the methods is known (e.g., SMN1/SMN2), but in some cases a signature variance is selected after sequencing a large number of samples in order to determine gene specific loci that are not affected by polymorphisms, gene conversions, or other genetic events. These gene specific loci will be used to accurately align NGS reads harboring at least one of these gene specific nucleotides.

In cases where there are 2 or more different gene-specific nucleotides between the genes, those differences may be employed in the method if they are within a certain number of bases (less than the length of NGS reads).

The methods provide carrier screen tests for individual(s) that are in need of determining whether or not they are a carrier for a genetic-based disease, including one in which the carrier would be autosomal recessive for a mutated gene in question. The individual may be male or female. In specific embodiments, the individual intends to procreate. The methods may be implemented as part of family planning for one or more individuals. The methods may or may not be employed as part of routine medical practices. The individual may be a pregnant female, such as one with an option of terminating a pregnancy dependent on the outcome of the carrier screen test. In addition, this method can also be used as a diagnostic test for individuals (fetus, infant, child or adult) who may be affected by such recessive diseases. Fetal tissues used for analysis may include CVS, amniocytes, or product of conception. The method may be employed as part of a single carrier testing assay that is for testing multiple genes or it may be a single gene testing assay or it may be used as part of multiple assays for multiple genes.

An individual may utilize the methods described herein as a sole user, or the methods may be performed by another party. In certain cases, an individual that utilizes the methods does so because of a desire for general personal genetic knowledge, because of family planning concerns, because of a concern for risk of producing offspring with SMA, or because of a known risk for producing offspring with SMA, for example because of family history or a positive result of another type of genetic test. The methods may be used as a primary and sole means of determining whether or not an individual is a carrier for SMA or may be used as a secondary means, such as obtaining a second opinion.

The disclosed methods may be utilized as a first tier test for determining whether or not an individual is a carrier for a genetic defect, which may be defined as carrier status. In specific embodiments, further testing to confirm whether or not an individual is a carrier may be employed, regardless of whether or not the individual tested as being a carrier or not being a carrier.

Although in particular embodiments the disclosed methods are employed to determine the copy number of SMN1 and SMN2 for carrier status for SMA, in some cases the carrier status for other genetic diseases may be queried. For example, one may determine the carrier status of congenital adrenal hyperplasia (CAH; CYP21A2/CYP21A1P), hemoglobin disorders (HBA1/HBA2), and any other genetic diseases that may be caused by gene copy number variations due to the presence of regions homologous to the disease genes.

In certain aspects, a sample is obtained from an individual in need of determining carrier/affected status for an allele. The sample from the individual may be of any kind so long as DNA is able to be extracted therefrom. The sample may be obtained using any method. In specific embodiments, the sample comprises blood, saliva, hair, semen, urine, feces, cheek scrapings, biopsy, amniotic fluid, chorionic villus, and so on.

### EXAMPLES

The following examples are included to demonstrate preferred embodiments of the invention. It should be appreciated by those of skill in the art that the techniques disclosed in the examples which follow present techniques discovered by the inventors to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1

### A Novel Algorithm for SMN1 and SMN2 Copy Number Analysis Using Coverage Depth Data from Next Generation Sequencing for the Detection of Spinal Muscular Atrophy (SMA) Carrier

Spinal muscular atrophy (SMA) is one of the most common autosomal recessive diseases with an incidence of ˜1 in 10,000 live births. The carrier frequency of this disease is approximately 1:40˜1:70 in different ethnic groups and population-based carrier screening is recommended by professional societies such as the ACMGG. SMA is caused by the complete loss of the survival motor neuron 1 (SMN1) protein while the number SMN2 copy gene may serve as a modifier for disease severity in affected patients. The underlying mechanism for SMN1 gene copy number change is attributed to its deletion or gene conversion. SMN1 and SMN2 are highly homologous with only five different nucleotides within the gene. The most important nucleotide that distinguishes SMN1 from SMN2 is located at +6 position in SMN1 exon 7 (c.840C>T in SMN2) acting as a transcription enhancer. Currently most clinical laboratories use quantitative assays (e.g. MLPA, qPCR) to analyze SMN1 copy numbers by interrogating the c.840C>T locus with or without other gene specific loci. In this work, there is provided a novel strategy using next generation sequencing (NGS) results from population carrier screening to analyze SMN1 copy number. After hybridization-based target enrichment and sequencing on an Illumina platform, a method was deployed that can accurately align sequence reads to SMN1 or SMN2. Gene specific reads were counted by surveying fragments with at least one of the SMN1/2 unique nucleotides in order to calculate SMN1:SMN2 copy number ratios. The total SMN1 and SMN2 copy numbers were independently determined by counting all of the exon 7 and neighboring exons' reads. Together with SMN1 and SMN2 total copy and their copy number ratio, SMN1 and SMN2 gene copy numbers were determined. Using this novel approach the inventors analyzed over 3,000 clinical samples and compared the copy number obtained from NGS with that from qPCR and/or MLPA studies. Individuals carrying one, two, three, four or above copies of SMN1 and SMN2 were all correctly identified by the NGS method. Potential limitations of this method due to gene hybrid or rare SNPs can be addressed by a refined local alignment algorithm and recounting gene specific reads. This method is useful to more efficiently perform large-scale carrier detection of SMA.

### Example 2

### Population Carrier Screening for SMA by NGS

The present example shows population carrier screening for spinal muscular atrophy by next generation sequencing.

Materials and Methods

DNA Samples—

The analyses were performed using de-identified samples collected for carrier testing according to protocols approved by the institutional review board at the Baylor College of Medicine. DNA was extracted from whole blood using commercially available DNA isolation kits (Gentra Systems, Minneapolis, Minn.) following the manufacturer's instructions.

Capture Enrichment and Next-Generation Sequencing—

A protocol previously described (Yang, et al., 2013) using capture-based target enrichment followed by NGS was adapted for the clinical test of 158 gene carrier sequencing. Briefly, genomic DNA samples were fragmented with the use of sonication, ligated to Illumina multiplexing paired-end adapters, amplified by means of a polymerase-chain-reaction assay with the use of primers with sequencing barcodes (indexes), and hybridized to biotin-labeled, solution-based capture reagent that was custom designed (Roche NimbleGen). Hybridization was performed at 47° C. for 64 to 72 hours, and paired-end sequencing (100 cycles each) was performed on the Illumina HiSeq.

NGS Data Processing and Copy Number Analysis—

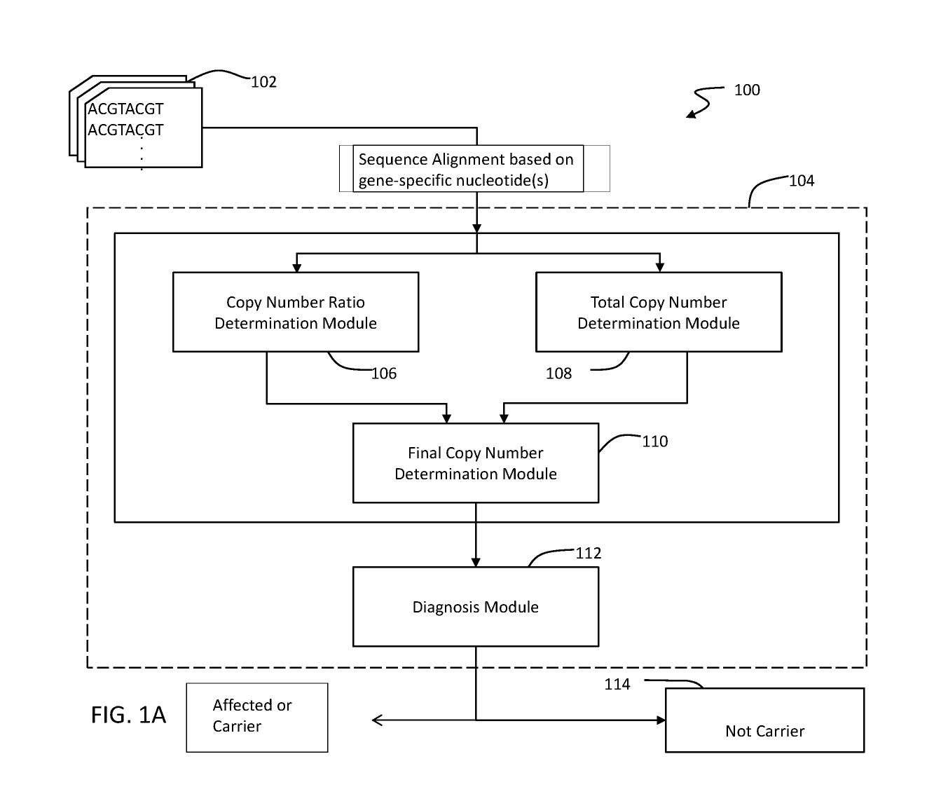

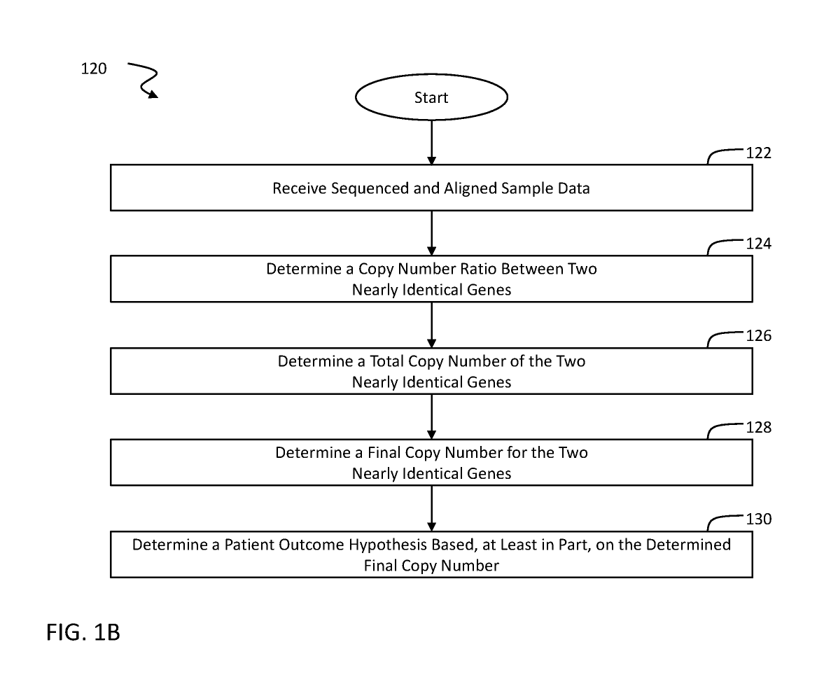

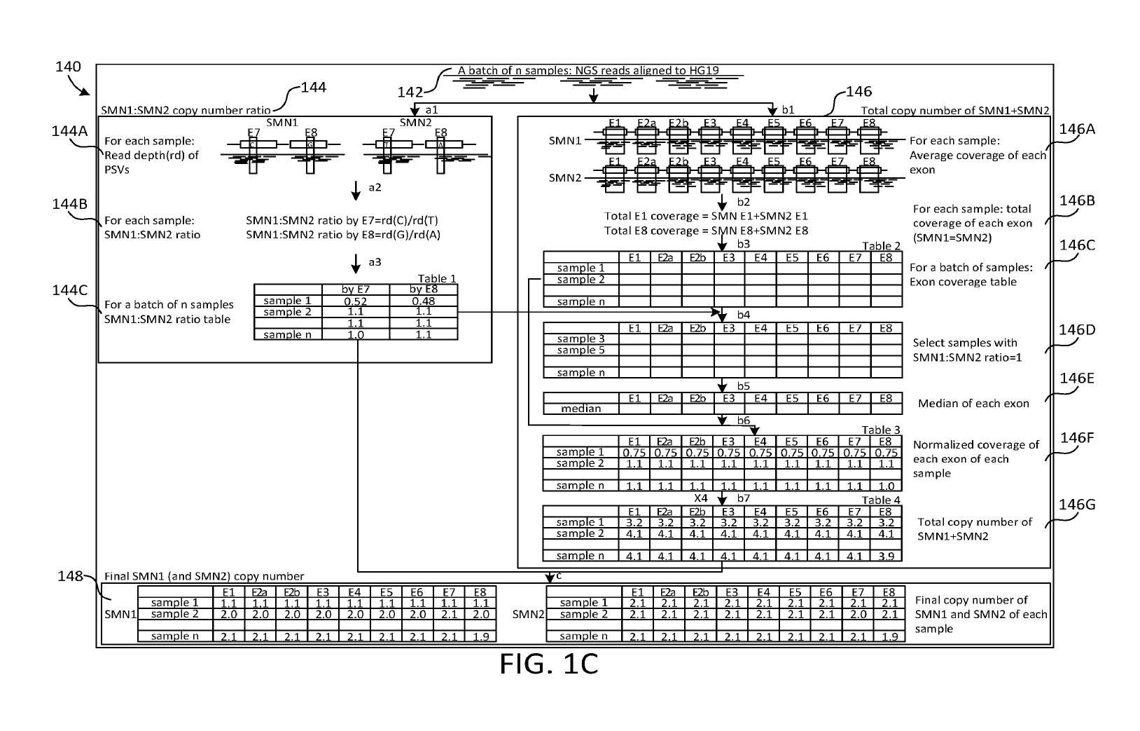

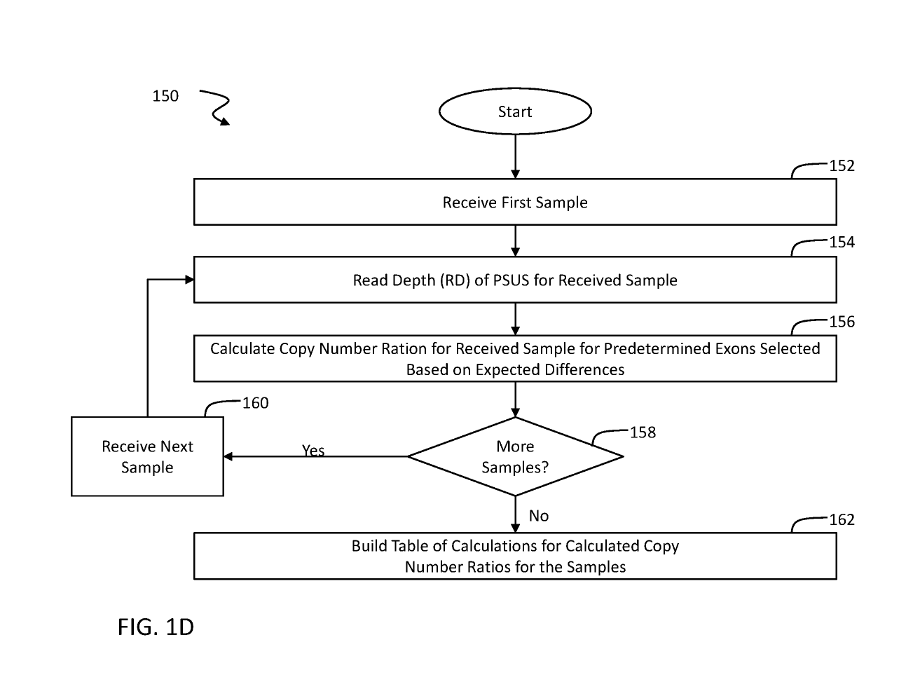

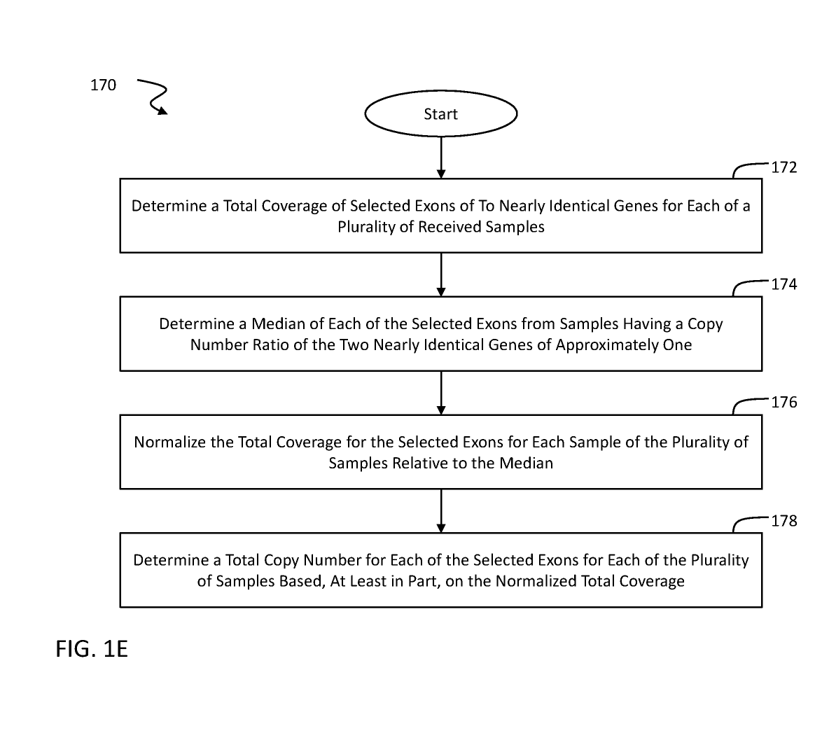

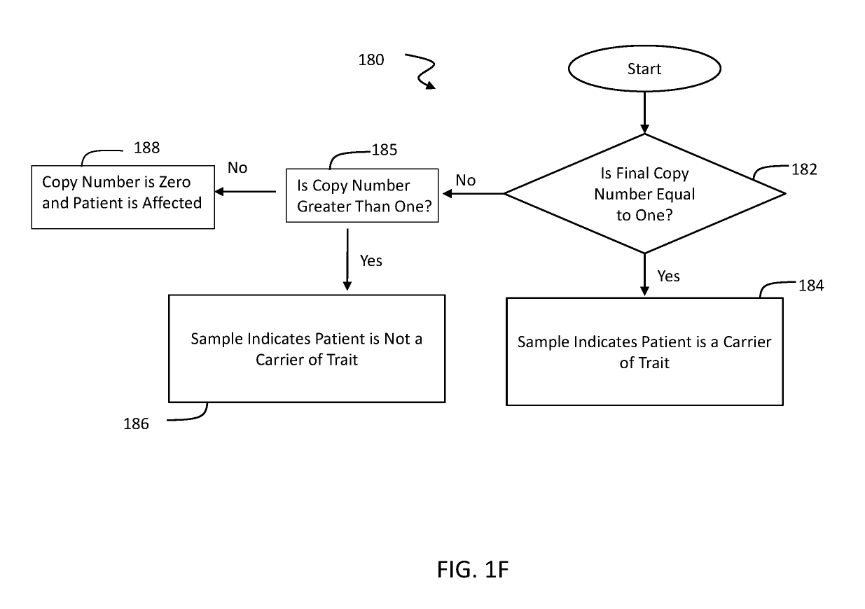

An example of NGS data processing and copy number analysis procedure is illustrated in FIGS. 1A-1F. In this example, samples from the same capture pool were grouped together. The raw sequence data can be aligned to hg19 reference by NextGENe software (available from SoftGenetics, State College, Pa.). Then, three steps may be performed in CNV analysis. A first step is to extract a read depth of the four PSV (paralogous sequence variant) loci of interest, in E7 and E8 of SMN1 and SMN2, and to calculate the copy number ratio of, e.g., SMN1 to SMN2, for each sample in the same capture pool. A second step is to generate the total (e.g., SMN1 and SMN2) copy number of each exon from the normalized average coverage depth of each exon according to CNV analysis algorithm (such as the one that is or is based on the one described in Feng, et al., 2015; Retterer, et al., 2015, or one modified from those algorithms), such that only the read depth of samples with SMN1:SMN2 ratios between 0.8-1.2 from the first step are selected to generate the median coverage depth of each exon. The total coverage depth of each exon is then normalized against the corresponding medians of the group. Finally, the total copy numbers of SMN1+SMN2 of each exon were obtained by multiplying the normalized values with 4. In a third step, the copy numbers are generated for individual SMN1 and SMN2 genes from SMN1:SMN2 copy number ratio from the first step and the total SMN1+SMN2 copy number from the second step.

Additional details regarding the NGS data processing is described with specific reference to the embodiments shown in FIGS. 1A-1F. FIG. 1A is a block diagram illustrating a system for processing data to determine a diagnosis for a patient, such as to determine whether the patient is a carrier of a trait, according to one embodiment of the disclosure. A system 100 may correspond to a software program embodied as various modules on a non-tangible computer readable medium. In another embodiment, the system 100 may correspond to circuitry, including logic and memory, configured to perform the functions described. In yet another embodiment, the system 100 may correspond to a combination of hardware and software, such as when a general purpose processor is executing code to perform steps that accomplish the described functions.

The system 100 may receive one or more input files 102 that include sequenced sample data. The sequenced sample data may be received from DNA sequencing, such as Next-Generation Sequencing (NGS) or Third Generation Sequencing, and may be aligned in reference to the hg19 or hg38 human genome, as examples. The input files 102 may be processed by one or more modules, such as a copy number ratio determination module 106 and a total copy number determination module 108. A copy number ratio and a total copy number may be determined by the modules 106 and 108, respectively, and their outputs provided to a final copy number determination module 110. A final copy number may be determined and provided to diagnosis module 112, which generates a diagnosis based, at least in part, on the final copy number received from module 110. The diagnosis may also be based on other data, such as information about a patient that provided a sample and/or statistical data regarding other patients in a cohort. The diagnosis may be output to a user, such as shown in display 114 indicating whether a patient is determined to be a carrier or affected of a trait. The output may be provided, such as shown in a window on a computer system, but the output may also be provided verbally, through e-mail, text message, a web interface, a printed report, or any other type of communication.

A method for processing sequenced data to determine a patient diagnosis is described in FIG. 1B. A method 120 begins at block 122 with receiving aligned and sequenced sample data, such as NGS data for a batch of samples, in which the NGS data is aligned to human gene hg19, for example. Then, at block 124, a copy number ratio between two nearly identical genes is determined (in specific embodiments, the term nearly identical may refer to two genes that are greater than 95, 96, 97, 98, 99, 99.1, 99.2, 99.3, 99.4, 99.5, 99.6, 99.7, 99.8, or 99.9% in identity). At block 126, a total copy number of the two nearly identical genes is determined. Block 124 may be processed prior to block 126 from the data received at block 122. Next, at block 128, a final copy number for the two nearly identical genes may be determined based, at least in part, on the determined copy number ratio of block 124 and the determined total copy number of block 126. The final copy number of block 128 may be used, in part or in whole, to diagnose a patient. At block 130, a patient outcome hypothesis may be determined based, at least in part, on the determined final copy number. The patient outcome hypothesis may be a determination as to whether a patient is a carrier of a genetic trait or other characteristic. That patient outcome hypothesis may be confirmed by other tests, such as to eliminate or reduce the likelihood of false positives or false negatives.

In one embodiment, the patient diagnosis systems and methods described above may be implemented specifically on the two nearly identical genes labeled the SMN1 and SMN2 genes (merely as examples). FIG. 1C is a block diagram illustrating a process for diagnosing whether a patient is a carrier of a trait related to the SMN1 and SMN2 genes. A data flow 140 may begin with receiving a batch of n samples when an NGS reads data aligned to hg19. That data may be processed in first data processing 144 and second data processing 146. The first data processing 144 may be used to determine a copy number ratio for SMN1:SMN2 genes. Processing 144 may include at processing block 144A reading depth (rd) of PSVs for each sample, then at block 144b determining a SMN1:SMN2 ratio for each sample. Several ratios may be computed, including an SMN1:SMN2 ratio given by E7=rd(C)/rd(T), and an SMN1:SMN2 ratio given by E8=rd(G)/rd(A). Next, block 144C includes building a table of the SMN1:SMN2 ratios for the batch of N samples received at block 142. Processing 146 may include, at processing block 146A, averaging a coverage of each exon for each sample. Then, at block 146B, there is calculation of a total coverage of each of some or all exons. For example, a total E1 coverage may be computed as SMN1+SMN2 E1, and a total E8 coverage may be computed as SMN E8+SMN2 E8. Next, at block 146C, an exon coverage table may be built for a batch of samples, and at block 146D samples selected that have a SMN1:SMN2 ratio equal to approximately one. A median or mean of each exon from the samples selected at block 146D is computed at block 146E. The exon coverage table of block 146C may then be normalized at block 146F, and a total copy number of SMN1+SMN2 computed at block 146G from the normalized coverage of block 146F. The total copy number of block 146G and the ratio table from block 144C may be combined to determine a final SMN1 and/or SMN2 copy number. Sample data for the various processing blocks is shown throughout FIG. 1C.

Referring back to the copy number ratio determination module 106 of FIG. 1A, the step of determining a copy number ratio between two nearly identical genes of block 124 of FIG. 1B, and processing block 144 of FIG. 1C, one specific calculation for a copy number ratio is shown in the embodiment of FIG. 1D. A method 150 for determining a copy number ratio begins at block 152 with receiving a first sample and then reading a depth (rd) of PSVs for the received sample at block 154. Next, a copy number ratio is calculated for the received sample for a predetermined set of exons, which may include some or all exons, wherein the predetermined exons may be selected based on having expected differences. At block 158, it is determined whether additional samples exist to process. If so, the next sample is received at block 160 and the processing returns to block 154. If not, a table may be built from the calculations of copy number ratios for the samples from the calculations of block 156.

Referring back to the total copy number determination module 108 of FIG. 1A, the step of determining a total copy number of the two nearly identical genes of block 126 of FIG. 1B, and processing block 146 of FIG. 1C, one specific calculation for a total copy number is shown in the embodiment of FIG. 1E. A method 170 may begin at block 172 with determining a total coverage of selected exons of two nearly identical genes for each of a plurality of received samples. Then, at block 174, a median may be determined for each of the selected exons from samples having a ratio of the two nearly identical genes equal to approximately one. Next, at block 176, the total coverage of block 174 may be normalized relative to all samples of the plurality of samples. Then, at block 178, a total copy number may be determined for each of the selected exons for each of the plurality of samples based, at least in part, on the normalized total coverage of block 176.

Referring back to the diagnosis module 112 of FIG. 1A, the step of determining a patient outcome hypothesis of block 130 of FIG. 1B, and the final copy number block 148 of FIG. 1C, one specific determination method for diagnosing a patient is shown in the embodiment of FIG. 1F. A method 180 begins at block 182 with determining of the final copy number. If the final copy number is one, the method proceeds to block 184 with the determination that the patient is a carrier of a trait. If not equal to one, the method 180 proceeds to block 185 to determine if the copy number is greater than one. If the copy number is greater than one, the method 180 proceeds to block 186 to determine that the sample indicates the patient is not a carrier of a trait. If the copy number is not greater than one, then the method 180 proceeds to block 188 to determine that the copy number is zero and the sample indicates the patient is affected for the trait.

The schematic flow chart diagrams of FIGS. 1A-1F is each generally set forth as a logical flow chart diagram. As such, the depicted order and labeled steps are indicative of aspects of the disclosed method. Other steps and methods may be conceived that are equivalent in function, logic, or effect to one or more steps, or portions thereof, of the illustrated method. Additionally, the format and symbols employed are provided to explain the logical steps of the method and are understood not to limit the scope of the method. Although various arrow types and line types may be employed in the flow chart diagram, they are understood not to limit the scope of the corresponding method. Indeed, some arrows or other connectors may be used to indicate only the logical flow of the method. For instance, an arrow may indicate a waiting or monitoring period of unspecified duration between enumerated steps of the depicted method. Additionally, the order in which a particular method occurs may or may not strictly adhere to the order of the corresponding steps shown.

If implemented in firmware and/or software, functions described above may be stored as one or more instructions or code on a computer-readable medium. Examples include non-transitory computer-readable media encoded with a data structure and computer-readable media encoded with a computer program. Computer-readable media includes physical computer storage media. A storage medium may be any available medium that can be accessed by a computer. By way of example, and not limitation, such computer-readable media can comprise random access memory (RAM), read-only memory (ROM), electrically-erasable programmable read-only memory (EEPROM), compact disc read-only memory (CD-ROM) or other optical disk storage, magnetic disk storage or other magnetic storage devices, or any other medium that can be used to store desired program code in the form of instructions or data structures and that can be accessed by a computer. Disk and disc includes compact discs (CD), laser discs, optical discs, digital versatile discs (DVD), floppy disks and Blu-ray discs. Generally, disks reproduce data magnetically, and discs reproduce data optically. Combinations of the above should also be included within the scope of computer-readable media.

In addition to storage on computer readable medium, instructions and/or data may be provided as signals on transmission media included in a communication apparatus. For example, a communication apparatus may include a transceiver having signals indicative of instructions and data. The instructions and data are configured to cause one or more processors to implement the functions outlined in the claims.

SMN1 and SMN2 Sequence Alignment—

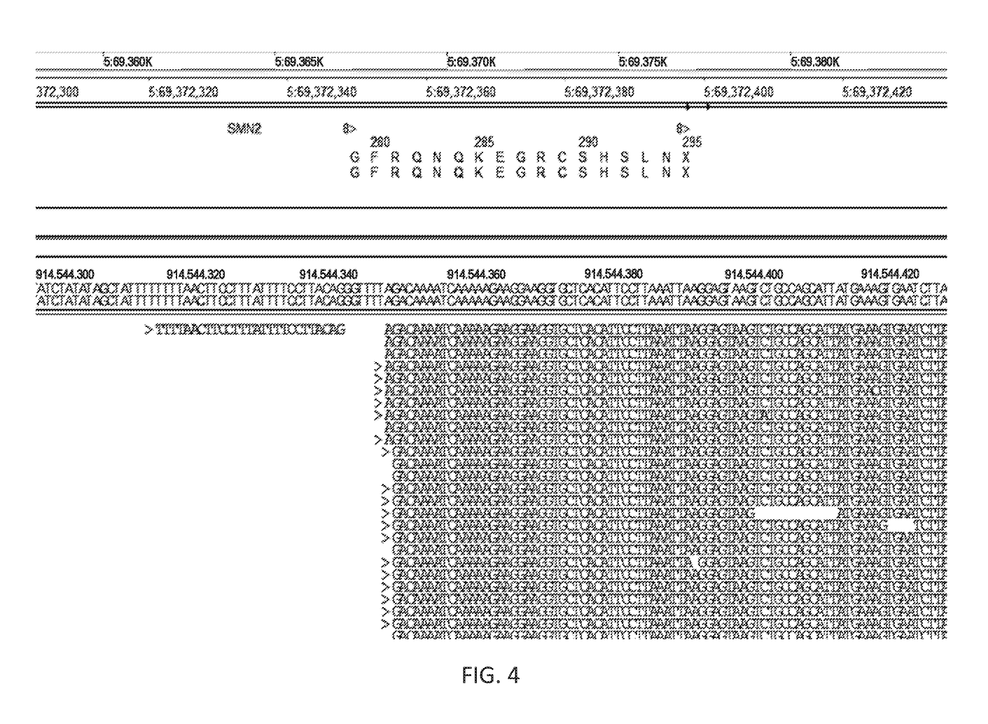

Because SMN1 and SMN2 only differ in five bases, the majority of SMN1 or SMN2 derived sequences are identical and cannot be distinguished by the aligner (a Burrows-Wheeler transform alignment method). As a result, these reads were ambiguously mapped to either SMN1 or SMN2 locus randomly with low mapping confidence. For any 100-bp read containing at least one SMN1 or SMN2 PSV, the aligner was able to map the reads to the reference correctly (FIG. 4). On the other hand, when none of PSVs was present in a given read, it would be misaligned. As illustrated in FIG. 4, all reads from a sample with two copies of SMN1 and zero copy of SMN2, mapped incorrectly to exons 7 and 8 of SMN2 were those without SMN2 PSVs. It was noticed that gene hybrids containing a single DNA fragment contains two PSVs, belonging to SMN1 and SMN2 or SNPs near the PSVs may confound the alignment. Therefore, the pair-end reads were decoupled and the alignment was performed based on single-end reads to increase mapping specificity.

SMN1 and SMN2 Copy Number Ratio—

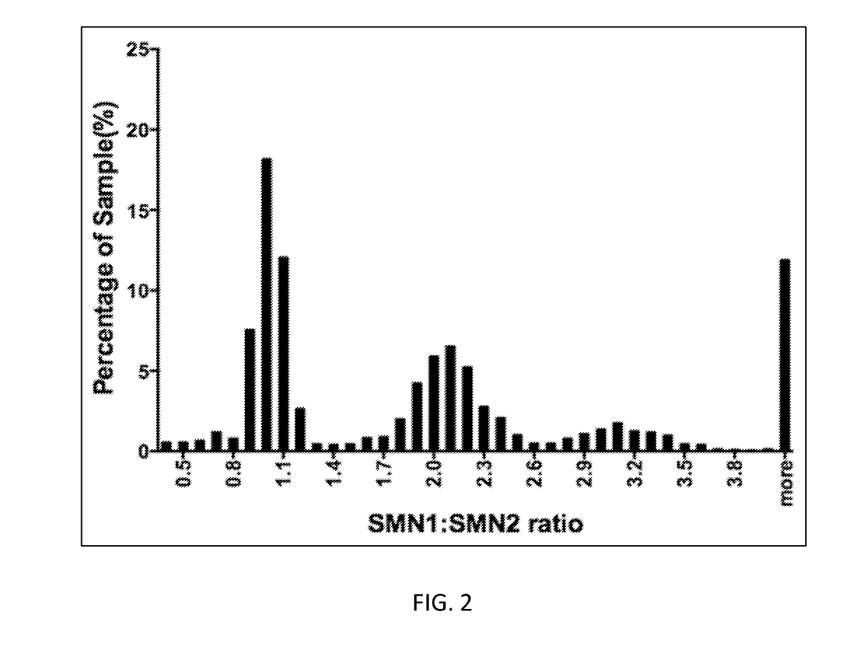

In order to utilize the NGS coverage data to analyze SMN1 or SMN2 copy number, It was considered that in any given sample the gene specific reads ratio should be directly determined by SMN1 and SMN2 gene copy number although the absolute reads number might be greatly affected by technical variations. To test this consideration, the SMN1:SMN2 copy number ratio was calculated of all samples in this study (n=2,488) by surveying all informative reads which harbor at least one of SMN1 or SMN2 PSVs. FIG. 2 demonstrates the copy number ratio distribution from the read depth of PSV on exon 7. Apparently, there are three major populations with the SMN1:SMN2 copy number ratio at 1, 2 or 3. This observation is in line with the fact that the most common configurations of SMN1 and SMN2 are individuals with 2 copies of SMN1 and 2 copies of SMN2; 2 copies of SMN1 and 1 copy of SMN2; 3 copies of SMN1 and 1 copy of SMN2.

SMN1 and SMN2 Copy Number Distribution—

Samples were grouped from the same capture pool to generate the total copy number of SMN1+SMN2 using previously published coverage based copy number analysis methods with modifications (Retterer, et al., 2015; Feng, et al., 2015). Briefly, the coverage of each exon of a test sample was compared to the value of the same exon in the reference file which is the median coverage of a group of samples. There are several modifications. First because SMN1 and SMN2 are highly homologous the NGS reads belonging to SMN1 or SMN2 may be misaligned in a random manner, so the coverage of the same exon from SMN1 and SMN2 were combined to generate the total SMN1+SMN2 copy number. Another modification is the reference file was not generated from all samples but from samples with SMN1:SMN2=1, because SMN1 or SMN2 copy number changes are common in our carrier samples and including too many samples with abnormal SMN1 or SMN2 copy number will compromise the quality of the reference file.

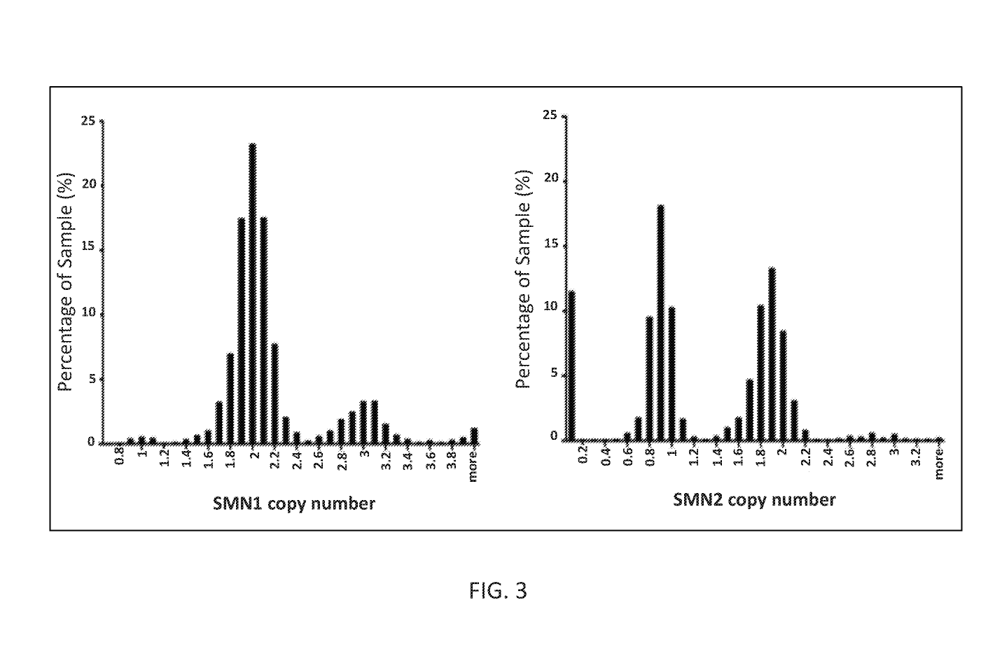

From the copy number ratio and total copy number of SMN1 and SMN2, one could determine copy number of individual SMN1 and SMN2 genes (FIG. 3). In the left panel, majority of the samples have 2 copies of SMN1. 3 copies of SMN1 are also common. There are 1.5% samples have 1 copy of SMN1, which shows a small peak at 1 in the figure. As for SMN2 copy numbers shown in the right panel, 1 copy and 2 copies are common in all samples. It is worth noting that 12% of all samples have 0 copy of SMN2.

The Test Sensitivity and Specificity of SMN1 Copy Number Detection from Capture NGS Data—

Batch affects test specificity in samples prepared from different capture pools were evident which introduced higher false positives, even when they were multiplexed together for sequencing in the same HiSeq flowcell (Table 1). Median coverage was used for each exon as an intra-batch normalizer for every capture pool library to calculate SMN1 and SMN2 copy numbers (FIG. 1). The inventors analyzed 2,488 clinical samples and compared the copy number obtained from capture NGS data with that from qPCR and/or MLPA studies (Table 1). For SMA carrier detection, the NGS test sensitivity is 100% (n=34, 95% confidence interval of 89.9-100%). The test specificity is 99.5% (n=2025, 95% confidence interval of 99.0-99.7%, Table 1). For detection of 3 copies and more SMN1, the NGS test sensitivity is 97.4% (n=420, 95% confidence interval of 95.4-98.5%). The test specificity is 99.6% (n=2,023, 95% confidence interval of 99.2-99.8%, Table 1).

TABLE 1
The test sensitivity and specificity of SMN1 copy number detection
by a novel computational algorithm from capture NGS data.
NGS test for SMA carriers (normalized per midpool)

| | Fluidigm/MLPA positive | Fluidigm/MLPA negative |
|---|---|---|
| NGS test positive | 34 | 11 |
| NGS test negative | 0 | 2014 |
| | | 95% CI |
| Sensitivity: | 100.0% | CI: 89.9-100% |
| Specificity: | 99.5% | CI: 99.0-99.7% |

NGS test for SMN1 3 copies and more (normalized per midpool)

| | Fluidigm/MLPA positive | Fluidigm/MLPA negative |
|---|---|---|
| NGS test positive | 409 | 9 |
| NGS test negative | 11 | 2014 |
| | | 95% CI |
| Sensitivity: | 97.4% | CI: 95.4-98.5% |
| Specificity: | 99.6% | CI: 99.2-99.8% |

The copy number calculation is more accurate if it is normalized by each midpool library, compared to that by each flowcell with two or more midpool libraries. Table 2 shows the sensitivity and specificity of SMN1 copy number detection when it is normalized for each flowcell with multiple midpool libraries. When it is for SMA carrier detection, the NGS test sensitivity is 100% (n=41, 95% confidence interval of 91.4-100%). The test specificity is 99.2% (n=2,274, 95% confidence interval of 98.7-99.5%, Table 2). For detection of 3 copies and more SMN1, the NGS test sensitivity is 94.6% (n=482, 95% confidence interval of 92.2-96.3%). The test specificity is 98.5% (n=2,290, 95% confidence interval of 97.9-98.9%, Table 2). Compared to the copy number data from Table 1, there is apparent improvement of sensitivity and specificity for SMN1 copy number detection when normalized by each midpool library, especially for 3 copies and more SMN1.

TABLE 2
The test sensitivity and specificity of SMN1 copy number
detection by a novel computational algorithm from capture NGS
data (normalized per flowcell).
NGS test for SMA carriers (normalized per flowcell)

| | Fluidigm/MLPA positive | Fluidigm/MLPA negative |
|---|---|---|
| NGS test positive | 41 | 19 |
| NGS test negative | 0 | 2255 |
| | | 95% CI |
| Sensitivity: | 100.0% | CI: 91.4-100.0% |
| Specificity: | 99.2% | CI: 98.7-99.5% |

NGS test for 3 copies and more (normalized per flowcell)

| | Fluidigm/MLPA positive | Fluidigm/MLPA negative |
|---|---|---|
| NGS test positive | 456 | 35 |
| NGS test negative | 26 | 2255 |
| | | 95% CI |
| Sensitivity: | 94.6% | CI: 92.2-96.3% |
| Specificity: | 98.5% | CI: 97.9-98.9% |

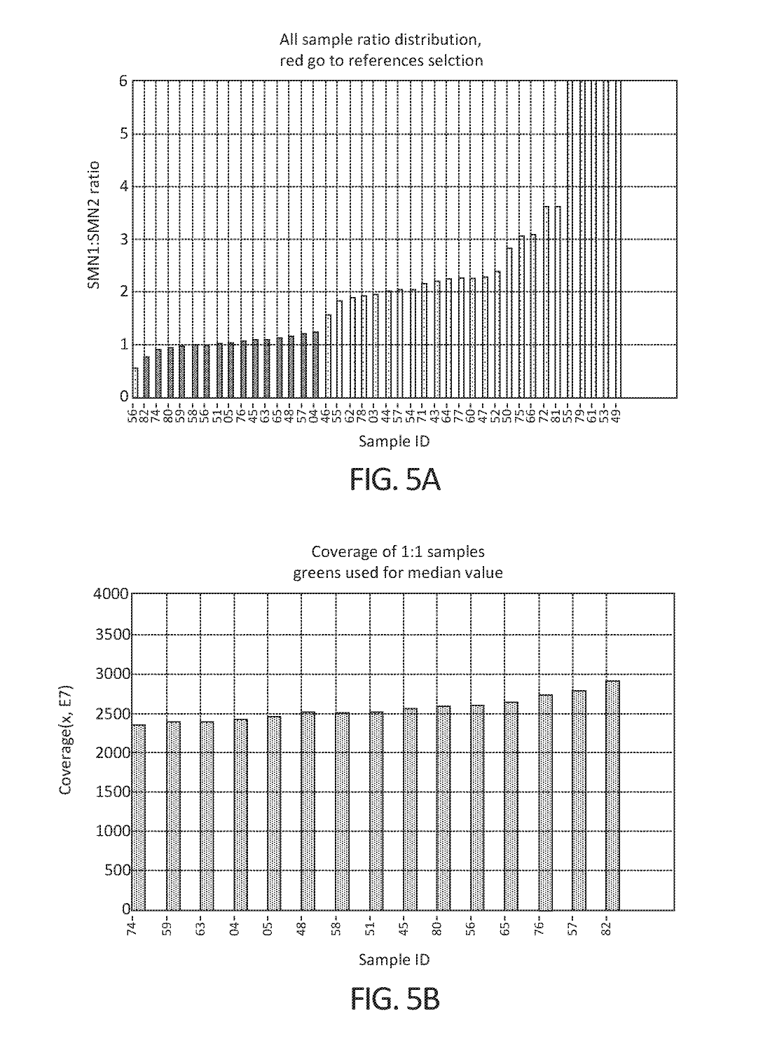

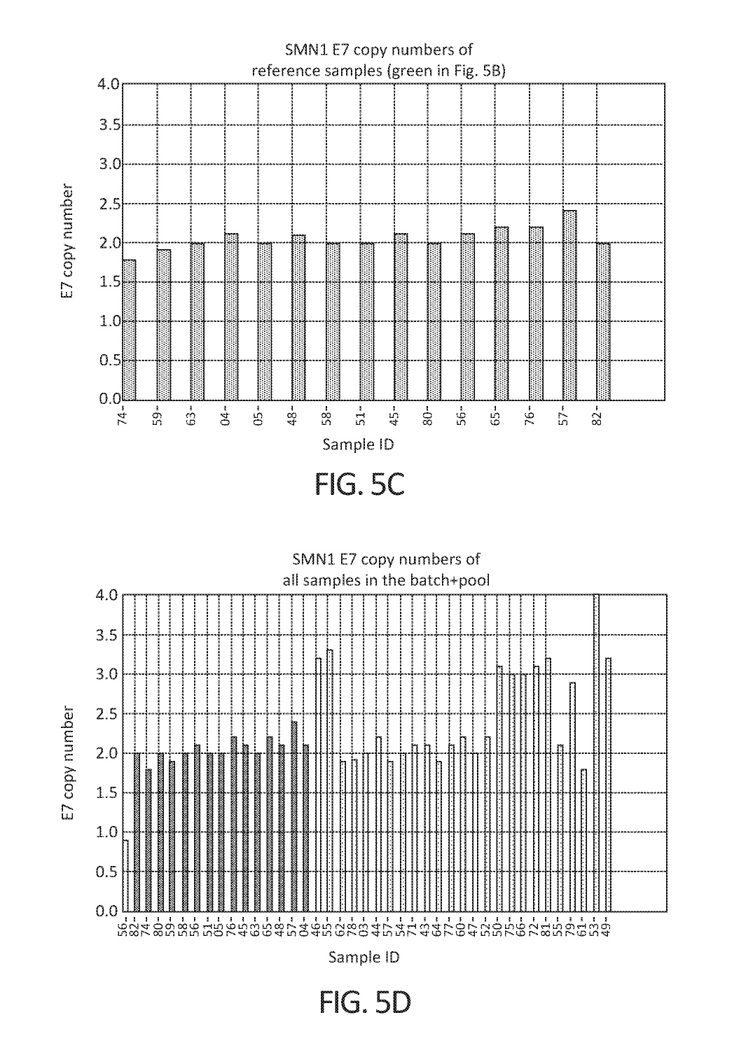

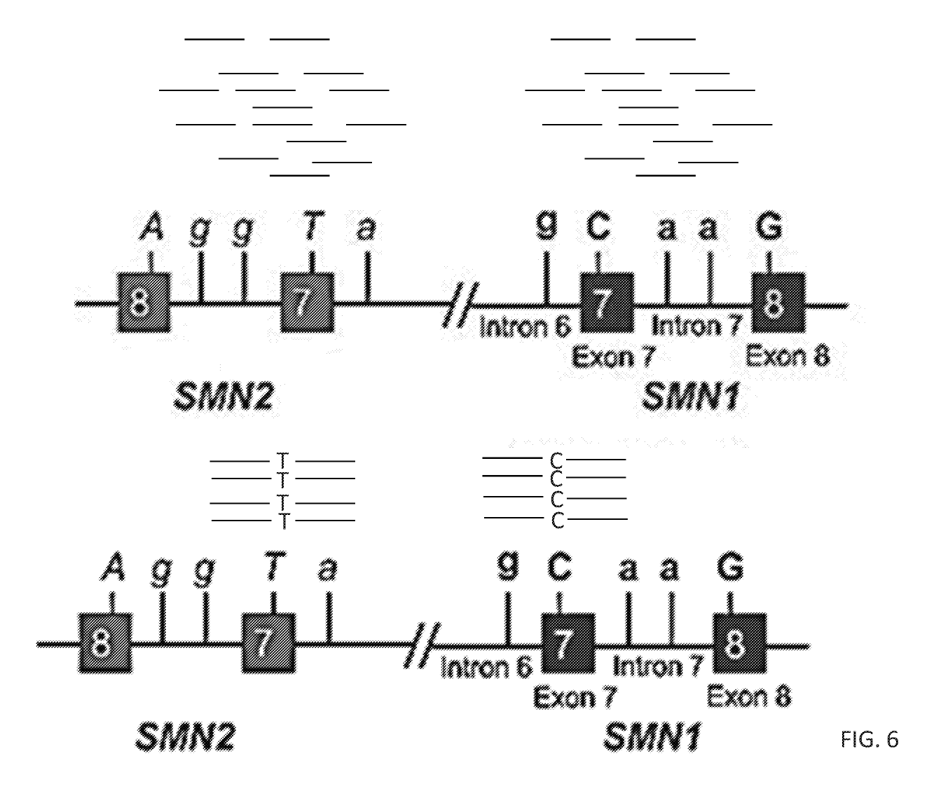

A diagram was generated consisting of four charts for visualization of SMN1:SMN2 ratio, coverage and final copy number calculation for all the samples in each batch (midpool library). The diagram provides an additional opportunity to manually check data quality in each batch. FIGS. 5A-5D shows a representative diagram for all the samples from a single midpool library. SMN1 Copy numbers are clearly shown in FIG. 5D, and there is clear separation of 1, 2, 3, 4 copies of SMN1.

### Significance of Certain Embodiments

NGS has made tremendous progress in clinical molecular testing including population carrier screening. While it generates reliable SNV results for a large number of genes in a high-throughput mode and can be used for CNV analysis, it is very challenging to generate reliable and reproducible CNV results for genes with highly homologous sequences, such as SMN1 and SMN2. In this study the inventors established and clinically validated a method that the exact copy numbers of SMN1 and SMN2 can be reliably obtained. First most NGS reads belonging to SMN1 or SMN2 may be mapped to either SMN1 or SMN2 randomly, NGS reads containing a PSV nucleotide can be accurately mapped to the correct locus with proper settings on the alignment. Therefore the read depth at the PSV position may represent the real coverage of the exon where the PSV is located, in specific embodiments. Subsequently, the read depth of such gene specific reads was used to calculate SMN1 to SMN2 copy number ratio. Because the majority of the NGS reads lack informative PSVs for accurate mapping, the coverage based on incorrectly aligned reads cannot be used for gene specific copy number analysis but can be useful for SMN1 and SMN2 total copy number analysis. Therefore, the inventors combined the non-discriminating reads from SMN1 or SMN2 together to obtain data to calculate their copy number. By taking this approach, there was maximization of the utility of coverage data in all exons of SMN1 and SMN2 that normally is discarded by routine NGS secondary analysis primarily designed for single gene mapping. Lastly, there was comparison of the copy numbers of SMN1 and SMN2 using the NGS based method of the disclosure with results from qPCR for over 2,488 samples, and comparable results were archived. Thus, provided herein is a highly sensitive and specific SMN1 copy number analysis method by NGS that is superior to conventional methods that are often affected by SNPs on the primer or probe binding sites. This SMA carrier testing method can be integrated to existing NGS based pan-ethnic carrier screening panels as a single test to add detection yields for SMA and reduce the overall cost.

Processing Systems for Processing Sequenced Data

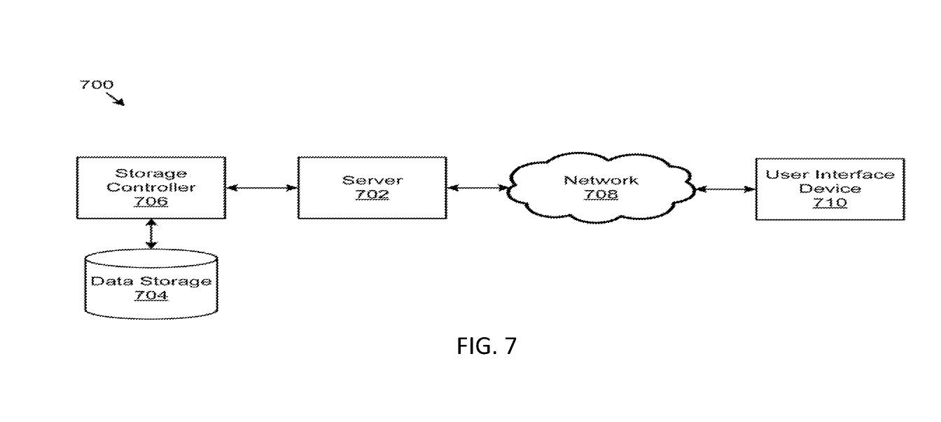

FIG. 7 illustrates one embodiment of a system 700 for multi-attribute clustering. The system 700 may include a server 702 and a data storage device 704. In a further embodiment, the system 700 may include a network 708 and a user interface device 710. In still another embodiment, the system 700 may include a storage controller 706 or storage server configured to manage data communications between the data storage device 704 and the server 702 or other components in communication with the network 708. In an alternative embodiment, the storage controller 706 may be coupled to the network 708. In a general embodiment, the system 700 may store databases comprising records, perform searches of those records, and calculate statistics regarding the records. In particular, the databases may store sequenced sample data and/or results of patient diagnoses.

In one embodiment, the user interface device 710 is referred to broadly and is intended to encompass a suitable processor-based device such as a desktop computer, a laptop computer, a Personal Digital Assistant (PDA), a mobile communication device or organizer device having access to the network 708. In a further embodiment, the user interface device 710 may access the Internet to access a web application or web service hosted by the server 702 and provide a user interface for enabling the service consumer (user) to enter or receive information, such as their diagnosis.

The network 708 may facilitate communications of data between the server 702 and the user interface device 710. The network 708 may include any type of communications network including, but not limited to, a direct PC-to-PC connection, a local area network (LAN), a wide area network (WAN), a modem-to-modem connection, the Internet, a combination of the above, or any other communications network now known or later developed within the networking arts which permits two or more computers to communicate, one with another.

The data storage device 704 may include a hard disk, including hard disks arranged in a Redundant Array of Independent Disks (RAID) array, a tape storage drive comprising a magnetic tape data storage device, an optical storage device, or the like. In one embodiment, the data storage device 704 may store health-related data, such as sequenced gene data, insurance claims data, consumer data, or the like. The data may be arranged in a database and accessible through Structured Query Language (SQL) queries, or other database query languages or operations.

FIG. 8 illustrates one embodiment of a database management system 800 configured to store and manage data for multi-attribute clustering. In one embodiment, the system 800 may include a server 702. The server 702 may be coupled to a data-bus 802. In one embodiment, the system 800 may also include a first data storage device 804, a second data storage device 806, and/or a third data storage device 808. In further embodiments, the system 800 may include additional data storage devices (not shown). In such an embodiment, each data storage device 804-808 may host a separate and/or redundant databases of healthcare information. Alternatively, the storage devices 804-808 may be arranged in a RAID configuration for storing redundant copies of the database or databases through either synchronous or asynchronous redundancy updates.

In one embodiment, the server 702 may submit a query to selected data storage devices 804-808 to collect a consolidated set of data elements associated with an individual or a group of individuals or organizations. The server 702 may store the consolidated data set in a consolidated data storage device 810. In such an embodiment, the server 702 may refer back to the consolidated data storage device 810 to obtain a set of data attributes associated with a specified sample. Alternatively, the server 702 may query each of the data storage devices 804-808 independently or in a distributed query to obtain the set of data elements associated with a specified individual. In another alternative embodiment, multiple databases may be stored on a single consolidated data storage device 810.

In various embodiments, the server 702 may communicate with the data storage devices 804-810 over the data-bus 802. The data-bus 802 may comprise a SAN, a LAN, or the like. The communication infrastructure may include Ethernet, Fibre-Chanel Arbitrated Loop (FC-AL), Small Computer System Interface (SCSI), and/or other similar data communication schemes associated with data storage and communication. For example, the server 702 may communicate indirectly with the data storage devices 804-810, the server first communicating with a storage server or storage controller 706.

The server 702 may host a software application configured for processing sequenced sample data, such as described in FIGS. 1A-1E. The software application may further include modules or functions for interfacing with the data storage devices 804-810, interfacing with a network 708, interfacing with a user, and the like. In a further embodiment, the server 702 may host an engine, application plug-in, or application programming interface (API). In another embodiment, the server 702 may host a web service or web accessible software application.

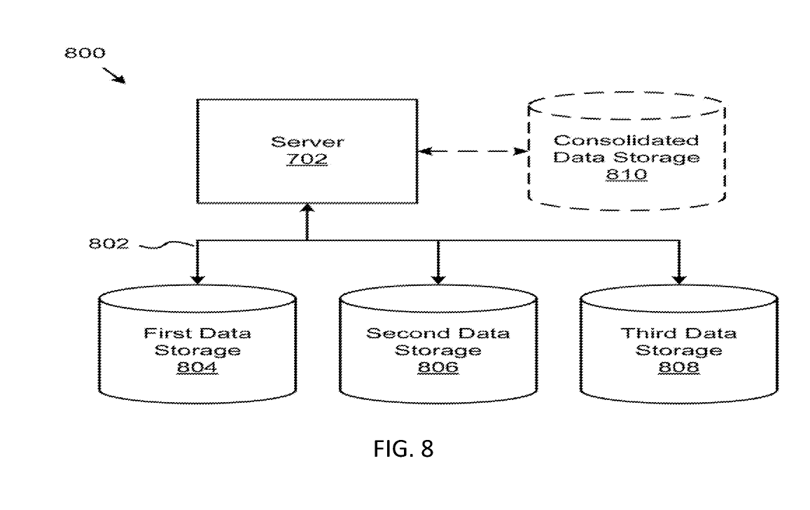

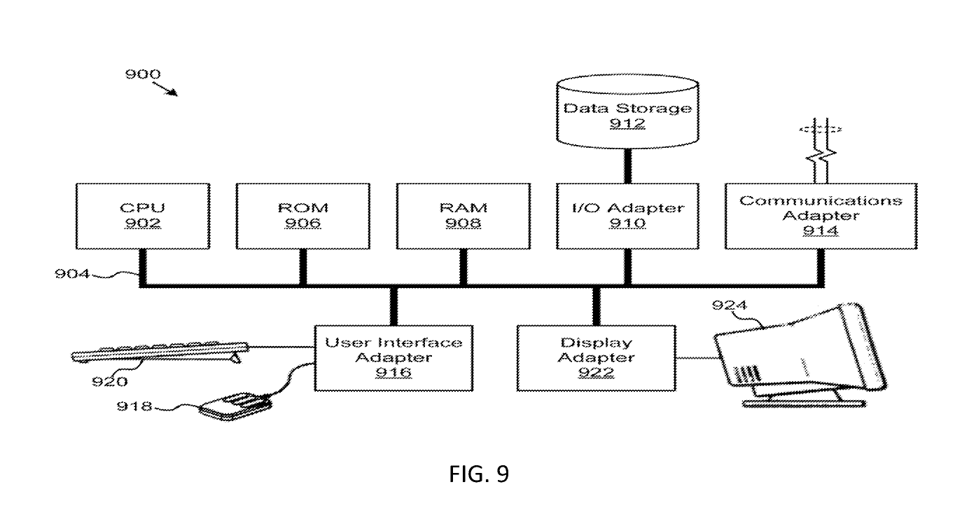

FIG. 9 illustrates a computer system 900 adapted according to certain embodiments of the server 702 and/or the user interface device 710. The central processing unit (CPU) 902 is coupled to the system bus 904. The CPU 902 may be a general purpose CPU or microprocessor. The present embodiments are not restricted by the architecture of the CPU 902, so long as the CPU 902 supports the modules and operations as described herein. The CPU 902 may execute the various logical instructions according to the present embodiments. For example, the CPU 902 may execute machine-level instructions according to the exemplary operations described above with reference to FIGS. 1A-1E.

The computer system 900 also may include Random Access Memory (RAM) 908, which may be SRAM, DRAM, SDRAM, or the like. The computer system 900 may utilize RAM 908 to store the various data structures used by a software application. The computer system 900 may also include Read Only Memory (ROM) 906 which may be PROM, EPROM, EEPROM, or the like. The ROM may store configuration information for booting the computer system 900. The RAM 908 and the ROM 906 may hold user and system 800 data.

The computer system 900 may also include an input/output (I/O) adapter 910, a communications adapter 914, a user interface adapter 916, and a display adapter 922. The I/O adapter 910 and/or user the interface adapter 916 may, in certain embodiments, enable a user to interact with the computer system 900 in order to input information for authenticating a user, identifying an individual, or receiving health profile information. In a further embodiment, the display adapter 922 may display a graphical user interface associated with a software or web-based application for processing sequenced sample data.

The I/O adapter 910 may connect one or more storage devices 912, such as one or more of a hard drive, a Compact Disk (CD) drive, a floppy disk drive, a tape drive, to the computer system 900. The communications adapter 914 may be adapted to couple the computer system 900 to the network 808, which may be one or more of a LAN and/or WAN, and/or the Internet. The user interface adapter 916 couples user input devices, such as a keyboard 920 and a pointing device 918, to the computer system 900. The display adapter 922 may be driven by the CPU 902 to control the display on the display device 924.

The present embodiments are not limited to the architecture of system 900. Rather the computer system 900 is provided as an example of one type of computing device that may be adapted to perform the functions of server 802 and/or the user interface device 810. For example, any suitable processor-based device may be utilized including without limitation, including personal data assistants (PDAs), computer game consoles, and multi-processor servers. Moreover, the present embodiments may be implemented on application specific integrated circuits (ASIC) or very large scale integrated (VLSI) circuits. In fact, persons of ordinary skill in the art may utilize any number of suitable structures capable of executing logical operations according to the described embodiments.

### Example 3

### The Next-Generation of Population-Based Spinal Muscular Atrophy Carrier Screening: Comprehensive Pan-Ethnic SMN1 Copy Number and Sequence Variant Analysis by Massively Parallel Sequencing

Introduction

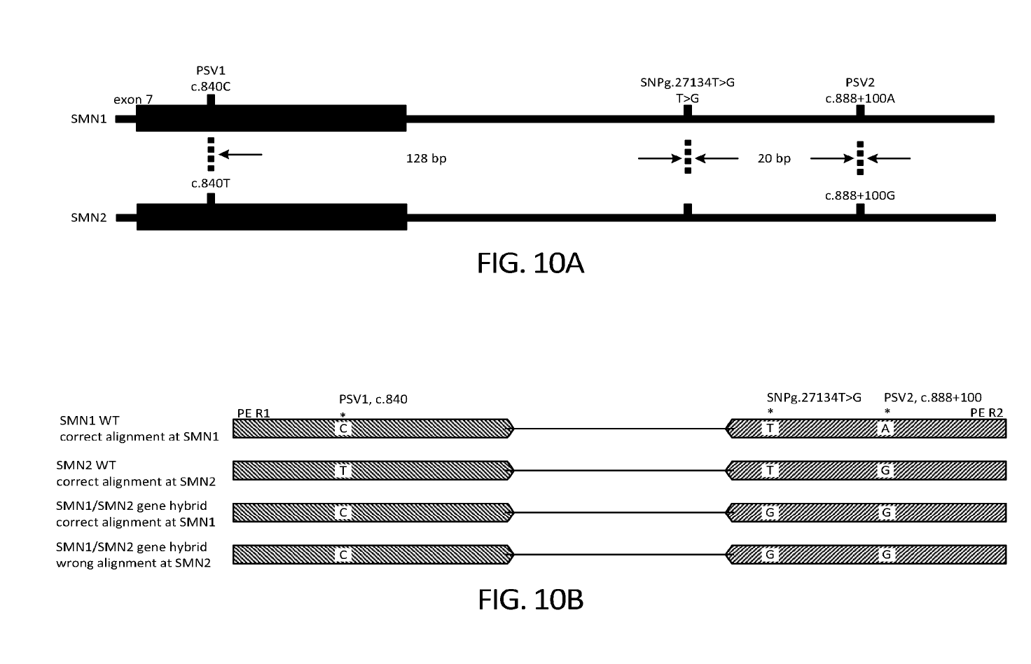

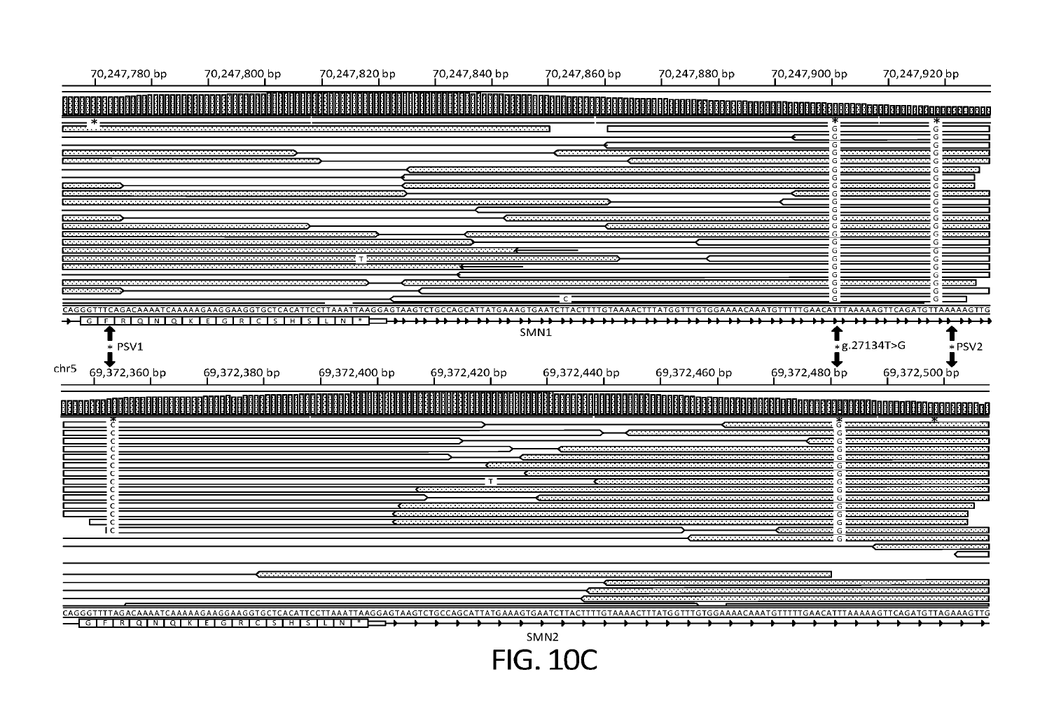

Spinal muscular atrophy (SMA, MIM #253300) is a neuromuscular disorder caused by loss of motor neurons in the spinal cord and brainstem, leading to generalized muscle weakness and atrophy that impairs activities such as crawling, walking, sitting up, and controlling head movement (Emery, et al., 1976). SMA has variable expressivity with a broad range of onset and severity. In severe cases, death occurs within the first two years of life mostly due to respiratory failure (Dubowitz, 1995). It has an incidence of about 1 in 10,000 live births and a carrier frequency of about 1/40 to 1/100 in different ethnic groups, with a higher carrier frequency in Caucasians and lower carrier frequencies in African Americans and Hispanics (Swoboda, et al., 2005; Hendrickson, 2009; Prior, et al., 2008; MacDonald, et al., 2014). SMA is caused by bi-allelic mutations in the survival motor neuron 1 (SMN1) gene including deletions, gene conversions and intragenic mutations, while SMN2 copy number may modify disease severity (Feldkotter, et al., 2002). SMN1 and SMN2 are highly homologous differing in five base pairs, none of which changes the amino acid sequence. A single C to T change in SMN2 exon 7 (c.840C>T) affects an exonic splicing enhancer (ESE), which results in a reduction of full-length transcripts from SMN2 (Lorson, et al., 1999). This nucleotide is considered as the only functional paralogous sequence variant (Lindsay, et al., 2006) (PSV, FIG. 10A) and is what differentiates SMN1 from SMN2.

SMA has features that can be recognized clinically but molecular testing is typically required to confirm the diagnosis. PCR coupled with restriction fragment length polymorphism (RFLP) analysis is a commonly used diagnostic test for SMA (van der Steege, et al., 1995), but this method does not detect carrier status. The first carrier test for SMA developed in 1997 used a competitive PCR strategy for quantification of SMN1 copy number (McAndrew, et al., 1997). Since then, the development of higher throughput methods, such as MLPA or qPCR, has enabled SMA carrier screening on a population basis (Cusco, et al., 2002; Arkblad, et al., 2006). These methodologies determine SMN1 copy number by interrogating the c.840C/T functional PSV that distinguishes the two SMN genes.

Massively parallel sequencing (MPS) or next-generation sequencing (NGS) technologies have rapidly transformed medicine as a cost effective approach to detecting pathogenic variants in patients with genetic diseases on a genomic scale (Yang, et al., 2014). Recently developed NGS-based carrier screening panels offer increased detection rates relative to conventional genotyping in a high-throughput mode for a large number of genes (Hallam, et al., 2014; Abuli, et al., 2016). Additionally, NGS is now used on a clinical basis for the detection of copy number variants (CNVs) (Retterer, et al., 2015; Feng, et al., 2015). The ability to detect such pathogenic variants when performing carrier screening by NGS is particularly important for diseases in which a high percentage of pathogenic variants are CNVs, as is the case with SMA. However, NGS based CNV detection is challenging for deletions and duplications at the single exon or sub-exon level due to technical noise introduced by uneven coverage in regions with variable GC content, non-linear amplification by PCR, and/or inter-run variations caused by assay artifacts known as batch effects. Another major drawback of CNV analysis by short-read NGS is the lack of locus-specific computational programs for genes with highly homologous sequences that have poor mappability to the genome. These genes, including SMN1 and SMN2, are normally excluded from NGS variant calling and copy number analyses (Mandelker, et al., 2016). In addition, SMN1 and SMN2 often undergo gene conversion events leading to gene hybrids that harbor PSVs from both genes (Cusco, et al., 2001). This complicates CNV analysis by NGS and underscores the need for nuanced data analysis to avoid errors caused by misalignment and gene conversion. SMN1 copy number analysis using a Bayesian hierarchical model applied to the 1,000 genome database was recently reported (Larson, et al., 2015). This analysis characterized individuals as “likely”, “possibly”, or “unlikely” SMA carriers. However, an NGS based clinical method for copy number analysis of SMN1 and/or other genes with highly homologous sequences has not been reported in the literature to our knowledge.

Sequence variants including single nucleotide variants or other small deletions, insertions or indels in SMN1 are medically relevant but not routinely detected by existing SMA carrier testing approaches. A recent study identified a SNP (g.27134T>G) tightly linked to a haplotype in silent carriers who have two copies of SMN1 in tandem on one chromosome and zero copy on the other 2+0 in certain populations (ADDENDUM: 2016). Analysis of these known SNPs was recommended in a recent update on SMA carrier testing by the ACMG (ADDENDUM, 2016). In addition, while whole gene or exonic CNVs account for the majority of SMA disease alleles, approximately 2.5% of SMA pathogenic variants are point mutations (MacDonald, et al., 2014). These pathogenic single nucleotide variants are not detected by carrier testing methods that only interrogate the c.840 PSV.

We have developed a novel method named PGCNARS (paralogous gene copy number analysis by ratio and sum) for SMA carrier testing based on short-read NGS data. This method was rigorously validated in a clinical setting using 6,738 pan-ethnic samples and compared to results generated by MLPA or qPCR. In addition, the g.27134T>G SNP associated with 2+0 SMA carrier status and pathogenic SMN1 sequence variants were also analyzed.

Materials and Methods

DNA Samples—

The analyses were performed using de-identified samples submitted to Baylor Genetics laboratory for carrier testing for a panel of diseases including SMA by NGS, qPCR and MLPA with the approval from the Institutional Review Board at Baylor College of Medicine. DNA was extracted from whole blood using commercially available DNA isolation kits (Gentra Systems, Minneapolis, Minn.) following the manufacturer's instructions.

SMN1 Copy Number Analysis by MLPA—

Copy number analysis for SMN1 was performed using the MCR-Holland SALSA MLPA Kit P060-B2 (MRC Holland, Netherland) or custom designed MLPA reagents according to manufacturer's recommendations. The MLPA reagent contains sequence specific probes targeted to exons 7 and 8 of both SMN1 and SMN2 (Schouten, et al., 2002). The MLPA data were analyzed using Coffalyzer software (MRC Holland, Netherland).

SMN1 Copy Analysis by Taqman Quantitative PCR—

SMN1 copy number was assessed by Taqman quantitative PCR assay as part of a panel using the BioMark 96.96 Dynamic Array (Fluidigm, South San Francisco, Calif.). Exon 7 from both SMN1 and SMN2 genes were amplified by the following primer pair, 5′-ATAGCTATTTTTTTTAACTTCCTTTATTTTCC-3′ (SEQ ID NO:35) and 5′-TGAGCACCTTCCTTCTTTTTGA-3′ (SEQ ID NO:36). A probe that specifically targets the SMN1 PSV (FAM-TTGTCTGAAACCCTG [SEQ ID NO:37]) was used to detect SMN1, while SMN2 was blocked by probe that targets the SMN2 PSV (VIC-TTTTGTCTAAAACCC [SEQ ID NO:38]). Quantitative PCR was performed on the BioMark HD system (Fluidigm, South San Francisco, Calif.) as previously described with minor modifications (Forreryd, et al., 2014). Copy number was calculated using the ΔΔCt method by normalizing to the genomic reference of the case and to the batch reference within the chip (Liu, et al., 2004).

Capture Enrichment and Next-Generation Sequencing—

A protocol previously described (Yang, et al., 2013) using capture-based target enrichment followed by NGS was adapted for the clinical test of 158 genes including SMN1 selected for carrier testing. Briefly, genomic DNA was fragmented by sonication, ligated to Illumina multiplexing paired-end adapters, amplified by polymerase-chain-reaction with indexed (barcoded) primers for sequencing, and hybridized to biotin-labeled, custom-designed (Roche NimbleGen, Madison, Wis.) capture probes in a solution-based reaction. Hybridization was performed at 47° C. for at least 16 hours, followed by paired-end sequencing (100 bp) on the Illumina HiSeq 2500 platform with average coverage of >300× in the targeted regions.

NGS Data Processing and Data Quality Control—

Raw image data conversion and demultiplexing were performed following Illumina's primary data analysis pipeline using CASAVA v2.0 (Illumina, San Diego, Calif.). Low-quality reads (Phred score <Q25) were removed prior to demultiplexing. Batched samples from the same capture pool were grouped and processed together. Sequences were aligned to the hg19 reference genome by NextGENe software using the recommended standard settings for SNV and indel discovery (SoftGenetics, State College, Pa.). In every sample, the average coverage depth of each targeted exon of non-homologous genes was extracted and normalized according to our previously published methods (Feng, et al., 2015). Similar to Derivative Log Ratio Spread (DLRS) used in the quality assurance of aCGH data analysis, DRS (Derivative Ratio Spread) was used to quantify the coverage depth variation of each sample from the NGS data, which is defined below.

$$DRS = \sqrt{\frac{1}{2 N} {\sum }_{i = 1}^{N} {( {δ}_{i} - μ )}^{2}}$$

δ stands for the difference of normalized coverage ratio between two adjacent exons; μ is the mean of all δ; N is the total number of data points which is the number of total exons minus 1. A sample with DRS>0.1 is considered as not passing quality control and thus not included for the copy number analysis. The script for the detection of is deposited at https://sourceforge.net/projects/PGCNARS

Results

SMN1 and SMN2 NGS Sequence Alignment Based on the Functional PSV at c.840—

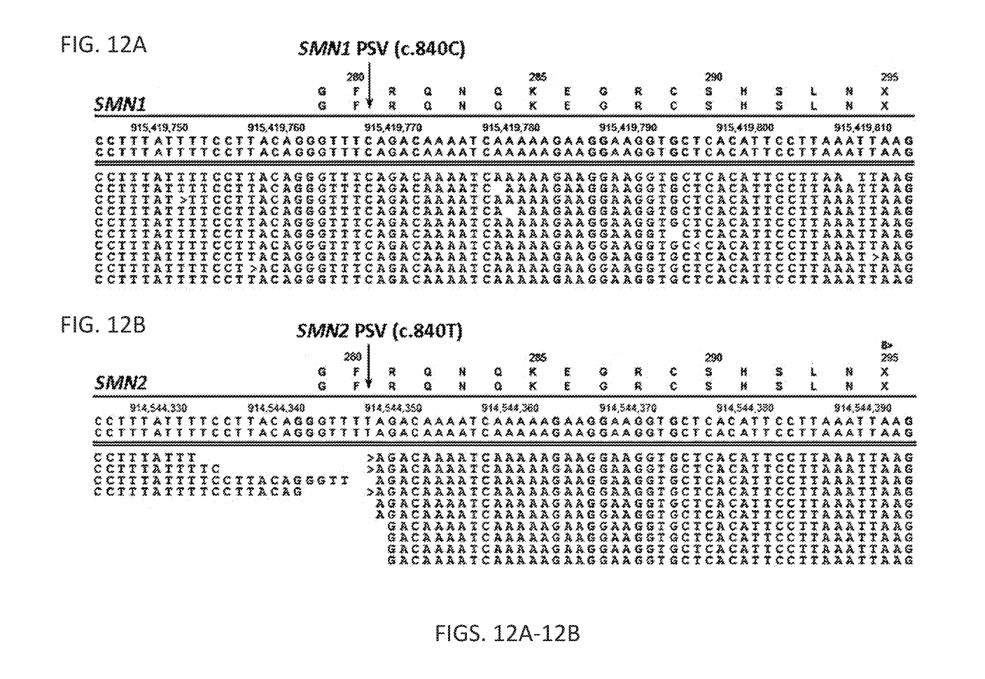

Since SMN1 and SMN2 differ at only five bases, most of the SMN1 or SMN2 derived NGS reads (2×100-bp pair-end sequencing used in this work) were indistinguishable. As a result, these reads were ambiguously aligned to either SMN1 or SMN2 with poor mapping quality, making read-depth-based copy number analysis inapplicable. Notably, reads containing at least one SMN1 or SMN2 PSV were mapped to the reference locus with higher mapping specificity. For example, in a sample with two copies of SMN1 and zero copy of SMN2 determined by MLPA, all correctly mapped NGS reads contained the SMN1 PSV (c.840C) in exon 7 (FIG. 12A). Reads that mapped incorrectly to exon 7 of SMN2 were those without the SMN1 PSV (FIG. 12B).

Effects of SMN1 and SMN2 Gene Conversion on Sequence Alignment and Read-Depth Analysis—

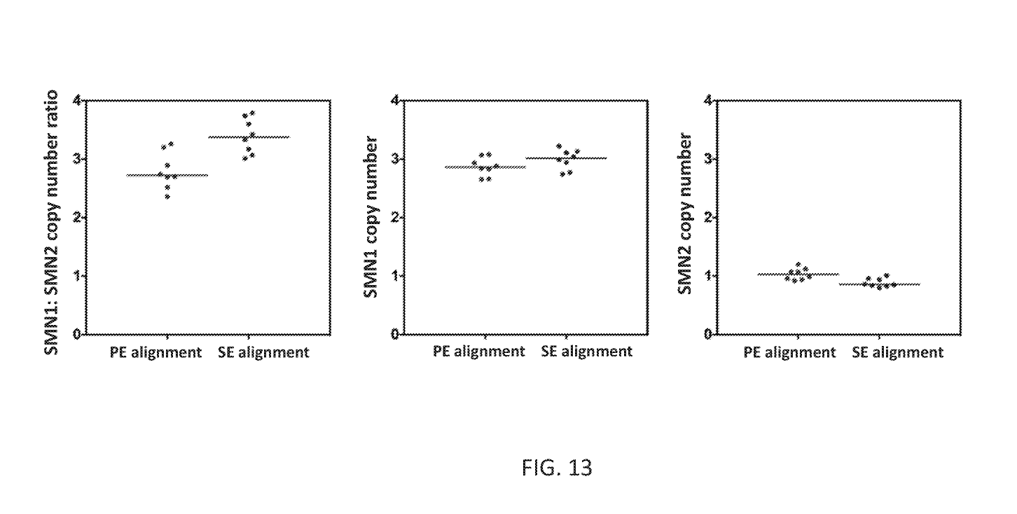

Since the functional PSV at c.840 is the only base which can be reliably used to differentiate the SMN1 and SMN2 genes, accurate read-depth data at this locus is necessary to determine the SMN1 and SMN2 copy number. However, gene conversions can produce SMN1 and SMN2 gene hybrids that harbor both SMN1 and SMN2 PSVs in a single SMN gene. In these samples, the SMN1 gene specific functional PSV (PSV1, c.840C in SMN1) and the SMN2 PSV (PSV2, c.888+100G in SMN2) can be found in a haplotype block containing exon 7 and intron 7 (FIG. 10A). The NGS reads derived from such gene hybrid regions may confound the mapping algorithm and result in incorrect alignment (FIG. 10B). For example, in a gene hybrid sample with the SMN1 functional PSV (c.840C), SMN1 SNP (g.271347T>G), and SMN2 PSV (c.888+100G) present in cis, 26% of the SMN1 sequences with the functional PSV mapped to the SMN2 locus (FIG. 10C). These SMN1 reads were misaligned to SMN2 because the pair-end (PE) read mapping algorithm did not always utilize the functional PSV c.840C to anchor the read pairs to the SMN1 locus when the SMN1 PSV c.840C was present on the 1^{st} read (R1) and the SMN2 intronic PSV and the SMN1 SNP were present on the 2^{nd} read (R2). Therefore, we decoupled the 2×100 PE reads and performed alignment based on single-end (SE) reads to achieve more accurate read-depth data at the SMN functional PSV locus. This was an essential step to correctly map reads containing the c.840C PSV to the SMN1 gene. We compared the performance of PE and SE alignment for eight gene-hybrid samples with three copies of SMN1 and one copy of SMN2 confirmed by MLPA. We found that SE mapping was more accurate for SMN gene copy number analysis. Compared to the SE alignment method, SMN1 to SMN2 copy number ratio was decreased and SMN1 copy number was underestimated by the PE alignment because some of the SMN1 reads were misaligned to the SMN2 locus (FIG. 13).

Calculation of SMN1 and SMN2 Copy Number by the Ratio and Sum of their NGS Reads—

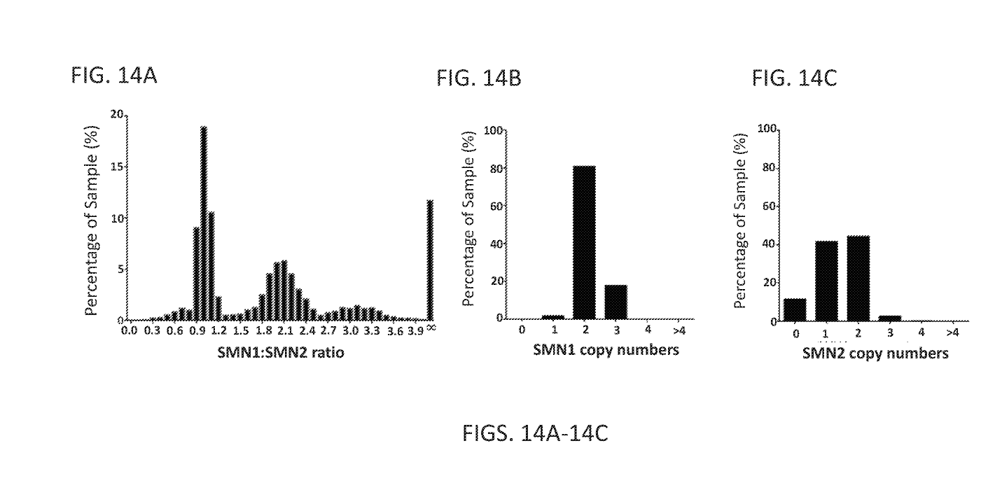

In order to determine SMN1 and SMN2 copy number using NGS data, we first hypothesized that in any given sample, the SMN1 to SMN2 copy number ratio should be determined by their gene specific reads ratio. To test this hypothesis, we calculated the SMN to SMN2 copy number ratio for all samples in this study (n=6,738) by surveying informative reads harboring the c.840C/T functional PSV in exon 7 or the c.*233T/A PSV in exon 8. The samples fell into three major populations with SMN1 to SMN2 copy number ratios of one, two or three (FIG. 14A). This observation was in line with the fact that the most common configurations of SMN1 and SMN2 include individuals with two copies of SMN1 and two copies of SMN2, two copies of SMN1 and one copy of SMN2, or three copies of SMN1 and one copy of SMN2 (Sugarman, et al., 2012; Contreras-Capetillo, et al., 2015; Sheng-Yuan, et al., 2010). Samples with zero copy of SMN2 were also relatively common (FIG. 14C). Samples with the same SMN1 and SMN2 gene copy number ratio frequently had different absolute gene copy numbers (e.g. individuals with two copies of SMN1 and SMN2 and those with three copies of each). Therefore, the copy number ratio itself could not be used directly to infer SMN1 and SMN2 copy number, but was informative only when it was used together with the combined SMN1 and SMN2 total copy number. We then calculated SMN1 and SMN2 total copy number using read-depth data by our previously published NGS based copy number analysis method with modifications (Feng, et al., 2015). We made an important adjustment to the published protocol which was to perform the analysis by capture batch. Samples pooled together in a single hybridization-based target enrichment reaction were analyzed and normalized as a group. This approach reduced the batch effects introduced by target capture, post-capture PCR, and sequencing variation. We observed a significantly higher error rate for SMN1 copy number calculations when samples from different capture pools were analyzed together, even when they were sequenced in the same flow-cell (Table 3).

TABLE 3
The comparison of two normalization methods within the
same target enrichment or sequencing group.

| | normalized by the | | normalized by the | |
|---|---|---|---|---|
| | median in samples | | median in samples | |
| | grouped in | | grouped in a | |
| | a capture pool | | flowcell | |
| | 1 copy | ≥3 copies | 1 copy | ≥3 copies |
| | SMN1 | of SMN1 | SMN1 | of SMN1 |
| Sensitivity | 100.0% | 98.2% | 100.0% | 94.6% |
| 95% | 95.9-100% | 97.3-98.8% | 91.4-100.0% | 92.2-96.3% |
| CI | (n = 90) | (n = 1168) | (n = 41) | (n = 481) |
| Specificity | 99.6% | 99.8% | 99.2% | 98.5% |
| 95% | 99.4-99.7% | 99.7-99.9% | 98.7-99.5% | 97.9-98.9% |
| CI | (n = 6648) | (n = 5570) | (n = 2274) | (n = 2290) |

To calculate SMN1 and SMN2 total copy number, we normalized exonic read-depth to total mapped reads of all targeted genes included in our carrier screening panel. All reads aligned to either SMN1 or SMN2 were counted in this step, including both gene-specific reads and those non-distinguishing reads lacking PSVs. Next, samples with SMN1 to SMN2 copy number ratios between 0.8-1.2 were grouped together to identify the median sample, which generally was a sample with two copies each of SMN1 and SMN2. The median sample served as an intra-batch SMN1 and SMN2 total read-depth normalizer for subsequent calculations. The exact SMN1 and SMN2 copy number of this normalizer was confirmed by MLPA or qPCR and demonstrated complete concordance with the NGS predicted value (i.e. two copies of SMN1 and SMN2) in >50 consecutive batches. Finally, the SMN1 copy number for each sample was determined by applying the following formula,

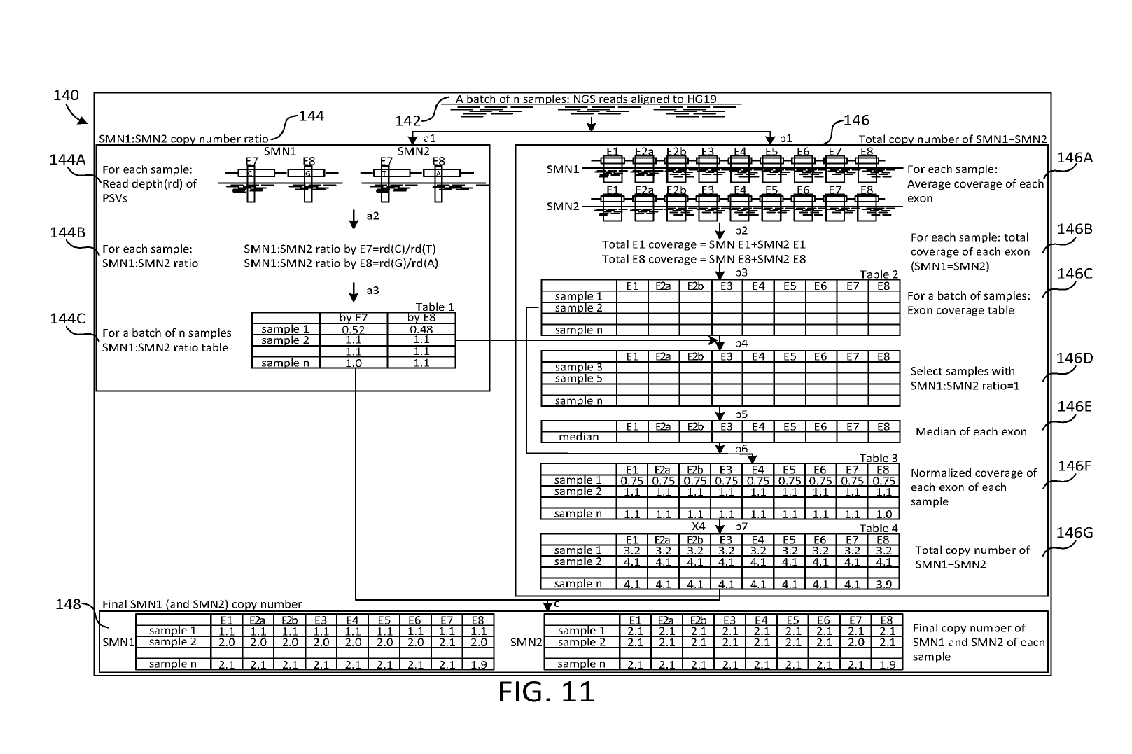

n1=rd1/(rd1+rd2)*Σc/χc*4

in which n1 is the calculated copy number of SMN1, rd1 and rd2 are the read depth of the c.840 PSV at SMN1 and SMN2 respectively, Σc is the combined exonic (exon 7) coverage of SMN1 and SMN2, and χc is the median of all the calculated Σc in a group of samples batched together for the analysis. The overall SMN1 and SMN2 copy number calculation algorithm is illustrated in FIG. 11. Note that the formula can also be used for the exon 8 copy number analysis to compare with the exon 7 copy number results by applying the coverage data of the exon 8 PSV (c.*233T/A). Using this method, we were able to differentiate SMA carriers who had one copy of SMN1 and SMN2 (1/1) from non-carriers who had two copies of each (2/2) although their SMN1 to SMN2 copy number ratios were not distinguishable (Table 4). For 1/1 and 2/2 individuals, they had an average of 2.1 and 3.98 total SMN1 and SMN2 copy number respectively. The same principle was applied to distinguish 1/2 carriers from a 2/3 carriers and/or other similar configurations.

TABLE 4
The ratios and frequencies different SMN1:t SMN2
copy number configurations.

| | | SMN1 to | SMN1 | | |
|---|---|---|---|---|---|
| | | SMN2 copy | and SMN2 | | |
| | SMN1:SMN2 | number | total | Frequency | |
| | copy number | ratio | copy number | (%) | n |
| Carriers | 1:0 | n/a | 1.17 | 0.05 | 3 |
| | | | (±0.20) | | |
| | 1:1 | 1.17 | 2.10 | 0.46 | 30 |
| | | (±0.091) | (±0.19) | | |
| | 1:2 | 0.53 | 3.02 | 0.90 | 59 |
| | | (±0.050) | (±0.25) | | |
| | 1:3 | 0.36 | 4.08 | 0.37 | 24 |
| | | (±0.041) | (±0.30) | | |
| Non- | 2:0 | n/a | 1.95 | 6.98 | 458 |
| carriers | | | (±0.17) | | |
| | 2:1 | 2.14 | 2.97 | 31.06 | 2038 |
| | | (±0.23) | (±0.19) | | |
| | 2:2 | 1.07 | 3.98 | 43.41 | 2848 |
| | | (±0.090) | (±0.23) | | |
| | 2:3 | 0.72 | 4.99 | 1.86 | 122 |
| | | (±0.060) | (±0.27) | | |
| | 3:0 | n/a | 3.04 | 2.73 | 179 |
| | | | (±0.17) | | |
| | 3:1 | 3.22 | 3.97 | 9.80 | 643 |
| | | (±0.31) | (±0.21) | | |
| | 3:2 | 1.61 | 5.04 | 2.16 | 142 |
| | | (±0.17) | (±0.28) | | |
| | 3:3 | 1.07 | 6.06 | 0.23 | 15 |
| | | (±0.11) | (±0.21) | | |

Mean and standard deviation are shown in the SMN1 to SMN2 copy number ratio and SMN1 and SMN2 total copy number columns.

Reproducibility, Sensitivity and Specificity of SMN1 Copy Number Analysis—

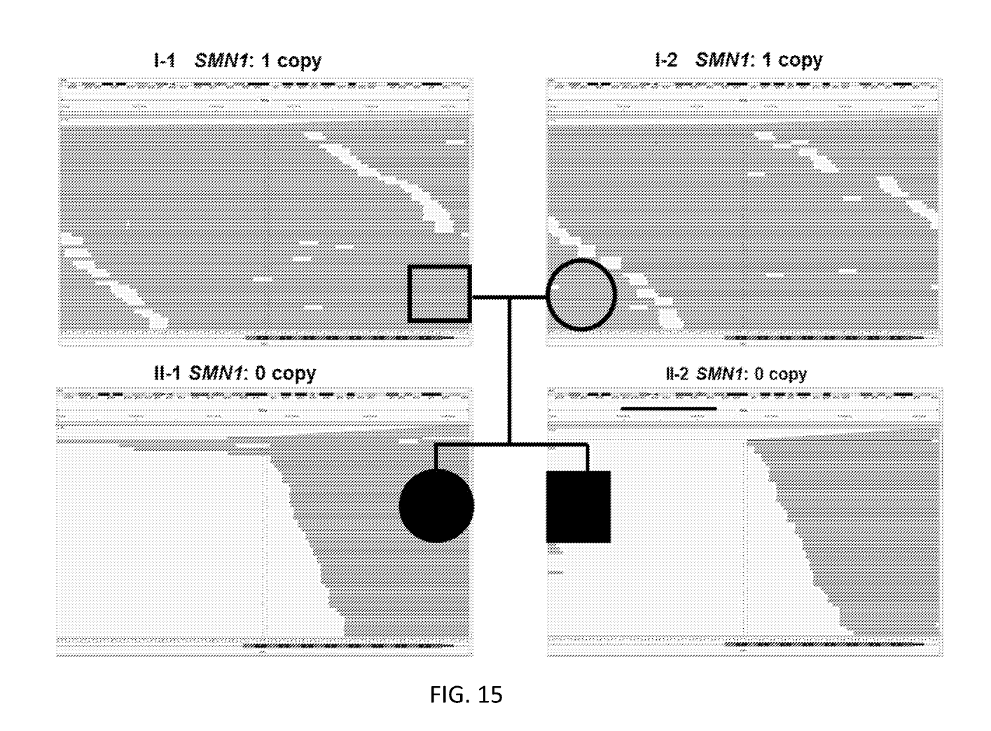

To determine the reproducibility of this new NGS based copy number analysis for SMN1, 68 samples were repeated in three independent runs among which 53 samples had two copies of SMN1, 11 had three or more copies of SMN1, and four had one copy of SMN1. This reproducibility test demonstrated complete concordance for all samples in all three runs. Next we analyzed 6,738 clinical samples submitted to our laboratory for carrier testing by comparing the qPCR and/or MLPA results to those generated by PGCNARS (Table 5). The test sensitivity was 100% for SMA carriers (95% CI, 95.9-100%, n=90) with a test specificity at 99.6% (95% CI, 99.4-99.7%, n=6,648). For samples with two copies of SMN1, the NGS method's test sensitivity and specificity were 99.4% (95% CI, 99.1-99.5%, n=5,480) and 98.3% (95% CI, 97.5-98.9%, n=1,258) respectively. For samples with three or more copies of SMN1, test sensitivity and specificity were 98.2% (95% CI, 97.3-98.8%, n=1,168) and 99.8% (95% CI, 99.7-99.9%, n=5,570) respectively. To test if the NGS-based SMN1 copy number analysis can be used for the diagnosis of SMA patients, we tested a familial tetrad in which two children were affected by SMA. Our NGS analyses showed that both of the affected children had zero copy of SMN1 while their parents were carriers with one copy of SMN1 (FIG. 15).

TABLE 5
The test sensitivity and specificity of SMN1 copy number
analysis by an NGS-based computational algorithm.

| | True positive | True negative |
|---|---|---|
| | confirmed by | confirmed by |
| SMN1 copy number | Fluidigm/MLPA | Fluidigm/MLPA |
| 1 copy of SMN1 | | |
| NGS test positive (1 copy of | 90 | 26 |
| SMN1) | | |
| NGS test negative (>1 copy of | 0 | 6622 |
| SMN1) | | |
| 2 copies of SMN1 | | |
| NGS test positive (2 copies of | 5445 | 21 |
| SMN1) | | |
| NGS test negative (1 or ≥3 copies | 35 | 1237 |
| of SMN1) | | |
| ≥3 copies of SMN1 | | |
| NGS test positive (≥3 copies of | 1147 | 9 |
| SMN1) | | |
| NGS test negative (<3 copies of | 21 | 5561 |
| SMN1) | | |
| | NGS | |
| | performance | 95% CI |
| 1 copy of SMN1 | | |
| sensitivity (n = 90) | 100.0% | 95.9-100% |
| specificity (n = 6648) | 99.6% | 99.4-99.7% |
| 2 copies of SMN1 | | |
| sensitivity (n = 5480) | 99.4% | 99.1-99.5% |
| specificity (n = 1258) | 98.3% | 97.5-98.9% |
| ≥3 copies of SMN1 | | |
| sensitivity (n = 1168) | 98.2% | 97.3-98.8% |
| specificity (n = 5570) | 99.8% | 99.7-99.9% |

Multiethnic SMN1 Copy Number Analysis for SMA Carrier Population Screening by NGS—

The multiethnic SMN1 copy number analysis data for SMA carrier population screening by NGS is summarized in Table 6. In 5,344 individuals with known ethnicity, African Americans and Hispanics had the lowest carrier frequency at 1.0% and 0.9% while Asians had the highest carrier frequency at 2.4%. Caucasians and individuals of Ashkenazi Jewish ancestry had SMA carrier frequencies at 1.4% and 1.9% respectively. About 47.8% of African Americans had three or more copies of SMN1 which is significantly higher than any other population. These results are consistent with previous studies of SMN1 copy number distribution in the general population⁴ indicating that the NGS method reported herein is robust in its determination of SMN1 copy number.

TABLE 6
The distribution of SMN1 copy number and g.27134T > G
SNP in different ethnic groups.

| | SMN1 one copy | | | | SMN1 two copies | | | | SMN1 three copies or more | | | | |
|---|---|---|---|---|---|---|---|---|---|---|---|---|---|
| | | | SNP + | Subtotal | | | SNP + | Subtotal | | | SNP + | Subtotal | |
| Ethnicity | SNP− | SNP+ | frequency | (frequency) | SNP− | SNP+ | frequency | (frequency) | SNP− | SNP+ | frequency | (frequency) | Total |
| Caucasian | 31 | 0 | 0.00 | 31 (0.014) | 1989 | 5 | 0.0025 | 1994 (0.917) | 132 | 18 | 0.12 | 150 (0.069) | 2175 |
| African | 12 | 2 | 0.14 | 14 (0.01) | 514 | 174 | 0.25 | 688 (0.511) | 138 | 506 | 0.79 | 644 (0.478) | 1346 |
| American | | | | | | | | | | | | | |
| Hispanic | 12 | 0 | 0.00 | 12 (0.009) | 1186 | 34 | 0.028 | 1220 (0.898) | 81 | 46 | 0.36 | 127 (0.093) | 1359 |
| Ashkenazi | 1 | 0 | 0.00 | 1 (0.019) | 46 | 0 | 0.00 | 46 (0.868) | 4 | 2 | 0.33 | 6 (0.113) | 53 |
| Jewish | | | | | | | | | | | | | |
| Asian^{a} | 10 | 0 | 0.00 | 10 (0.024) | 370 | 2 | 0.0054 | 372 (0.905) | 27 | 2^{b} | 0.069 | 29 (0.071) | 411 |
| Total | 66 | 2 | 0.03 | 68 (0.013) | 4105 | 215 | 0.05 | 4320 (0.808) | 382 | 574 | 0.60 | 956 (0.179) | 5344 |

SNP, SMN1 g.27134T > G analysis.
^{a}The Asian population included 146 East Asian, 98 South Asian, and 167 Southeast Asian individuals.
^{b}One South Asian and one Southeast Asian individual who have two copies of SMN1 and positive for the g.27134T > G SNP.

Detection of the g.27134T>G SNP Associated with 2+0 SMA Carrier Status by NGS—

Next we tested if our NGS assay could detect a recently identified g.27134T>G SNP associated with 2+0 SMA carrier status (Luo, et al., 2014). Our NGS method to call the g.27134T>G SNP yielded completely concordant results with those generated by an RFLP assay in 493 consecutive samples (Supporting Information and Supplementary Methods and Procedures). Importantly, using the NGS method we found that 574 of the 956 (79%) individuals with three or more copies of SMN1 were also positive for the g.27134T>G SNP while only 5% of individuals with two copies of SMN1 were carriers of the g.27134T>G SNP (Table 6). Therefore, testing for this SNP in the general population could theoretically identify 2+0 SMA carriers. In our cohort, linkage of the SNP with the SMN1 duplicated allele varied by ethnic group. Based on the configurations of SMN1 copy number and the g.27134T>G SNP genotype, we found linkage was the highest in African Americans; 65.9% of duplicated SMN1 alleles were also positive for the g.27134T>G SNP. Linkage was the lowest for Asians with a positive SNP frequency of 6.6% among the duplicated alleles. The linkage was 11.9%, 34.8% and 33.3% for Caucasians, Hispanics and Ashkenazi Jews respectively. When SMN1 copy number and g.27134T>G SNP analyses were combined to identify SMA carriers, the detection rate was increased to 85.9-95.3% in different ethnic groups compared to SMN1 copy number based carrier testing (Table 7). Therefore, the residual risk of being an SMA carrier after a negative screening result (i.e. two copies of SMN1 and negative for g.27134T>G SNP) decreases in all populations (Table 7). The positive prediction value for an individual to be a 2+0 carrier after testing positive for the g.27134T>G SNP with two copies of SMN1 is highest among Ashkenazi Jews (˜100%) but lower in other ethnic groups ranging from 1 in 174 to 1 in 40 (Table 7).

TABLE 7
SMA carrier detection and residual risk estimates.

| | | | Residual | Detection | Residual risk | Risk for |
|---|---|---|---|---|---|---|
| | Carrier | Detection | risk (CN | rate | (CN + SNP | SNP |
| Ethnicity | frequency | rate (CN) | negative) | (CN + SNP) | negative) | positive |
| Caucasian | 1 in 35^{a} | 94.9%^{a} | 1 in 668 | 95.3% | 1 in 724 | 1 in 40 |
| African | 1 in 66^{a} | 71.1%^{a} | 1 in 226 | 85.9% | 1 in 463 | 1 in 83 |
| American | | | | | | |
| Hispanic | 1 in 117^{a} | 90.6%^{a} | 1 in 1,235 | 92.2% | 1 in 1,488 | 1 in 174 |
| Ashkenazi | 1 in 41^{b} | 90.0%^{b} | 1 in 401 | 91.8% | 1 in 489 | carrier |
| Jewish | | | | | | |
| Asian | 1 in 53^{a} | 92.6%^{a} | 1 in 704 | 92.8% | 1 in 723 | 1 in 87 |

CN, SMN1 copy number analysis;
CN negative, two copies of SMN1 detected;
SNP, SMN1 g.27134T > G analysis;
CN + SNP negative, two copies of SMN1 detected and g.27134T > G not detected.
^{a}Reference 4.
^{b}Reference 22.

SMN1 Sequence Pathogenic Variants Identified by NGS—

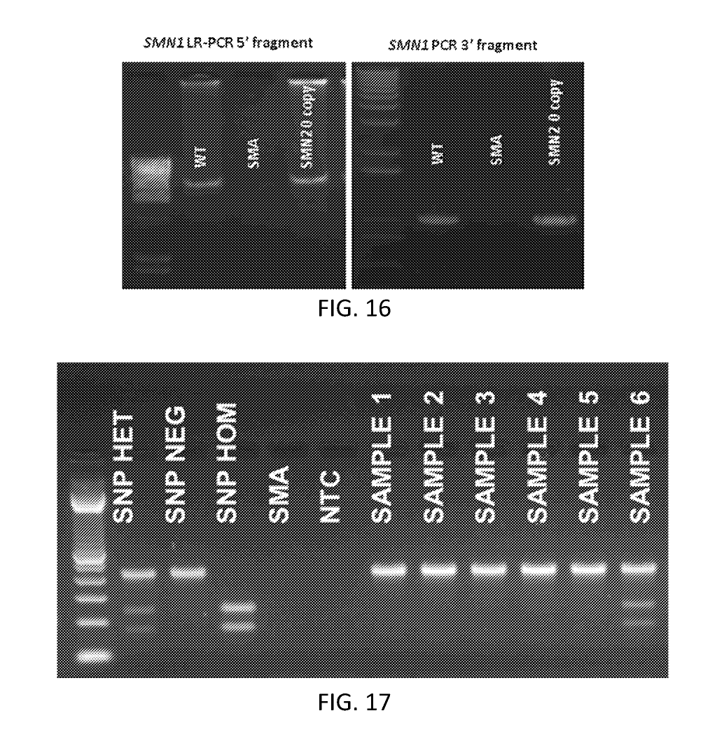

Among all samples analyzed for sequence variants by NGS, we identified ten individuals with potentially pathogenic single nucleotide variants in SMN1 gene. These variants were either previously found in SMA patients or novel likely pathogenic variants (Table 8). We confirmed the NGS results by using gene-specific PCR followed by amplicon-based sequencing (FIG. 16).

TABLE 8
The SMN1 variants identified by NGS screening and confirmed
by gene specific sequencing.

| Sample | Gene | Nucleotide | AminoAcid | Comments |
|---|---|---|---|---|
| 1 | SMN1 | c.154-1G > T | | Splice site |
| 2 | SMN1 | c.154-1G > T | | Splice site |
| 3 | SMN1 | c.346A > G | p.I116V | Allelic to SMN1:c. |
| | | | | 346A > T (I116F), |
| | | | | PMID: 15249625 |
| 4 | SMN1 | c.662C > T | p.P221L | PMID: 24498607 |
| 5 | SMN1 | c.662C > T | p.P221L | PMID: 24498607 |
| 6 | SMN1 | c.662C > T | p.P221L | PMID: 24498607 |
| 7 | SMN1 | c.662C > T | p.P221L | PMID: 24498607 |
| 8 | SMN1 | c.662C > T | p.P221L | PMID: 24498607 |
| 9 | SMN1 | c.662C > T | p.P221L | PMID: 24498607 |
| 10 | SMN1 | c.662C > T | p.P221L | PMID: 24498607 |

### Discussion

NGS has enabled tremendous progress in clinical molecular testing including population-based expanded carrier screening (Hallam, et al., 2014; Abuli, et al., 2016; Haque, et al., 2016). A recent large cohort study suggested that expanded carrier screen involving NGS increases detection rates for a variety of potentially serious genetic diseases when compared with current recommendations, which focus on testing a small number of diseases in high-risk populations (Haque, et al., 2016). While NGS generates reliable SNV results in a high-throughput mode and can be used for CNV analysis, calling sequence and copy number variants for genes with highly homologous sequences is technically challenging. For this reason, SMN1 and SMN2 have been put into a “dead zone” of genes that are not amenable to accurate NGS alignment (Mandelker, et al., 2016).

The majority of SMN1 and SMN2 NGS short reads lack informative PSVs for accurate mapping and simple depth of coverage analyses cannot be used directly for gene-specific copy number analysis. However, ambiguously aligned reads (i.e. reads aligned to SMN1 or SMN2) may be used to calculate the total combined copy number of SMN1 and SMN2. Gene-specific reads containing the c.840C/T PSV can then be used to calculate the SMN1 to SMN2 copy number ratio and in turn permit derivation of gene-specific copy number. We used this approach to analyze 6,738 samples submitted to our lab for carrier testing. Measures of test reproducibility, sensitivity, and specificity indicate that this NGS method is highly accurate and robust for SMN1 copy number analysis.

A recent study identified several SNPs, including g.27134T>G, which are tightly linked to a haplotype in 2+0 carriers who have two copies of SMN1 in tandem duplication on one chromosome and zero copy on the other (Luo, et al., 2014). Since our carrier screening panel was designed to analyze the entire coding sequence and flanking intronic regions of every gene on the panel, including SMN1 and SMN2, we were able to detect clinically relevant SMN1 sequence variants (e.g. g.27134T>G) in addition to copy number changes. We determined SMN1 copy number and genotyped the g.27134T>G SNP in different ethnic groups and found that this approach increases SMA carrier detection rates in all ethnic groups compared to conventional methodologies. The positive prediction value (PPV) for an individual to be a SMA carrier when SMN1 copy number is two the g.27134T>G SNP is present, is highest for Ashkenazi Jews (˜100%), which is consistent with the previous study (Luo, et al., 2014). The PPV was much lower for the general Asian population (˜1.1%), however, in contrast to the previous report (˜100%). This discrepancy could be due sampling differences as distinct Asian subpopulations were included in our study (Table 7). It should be noted that only a fraction of SMN1 duplicated alleles were linked to the g.27134T>G SNP in individuals other than African Americans, and further study will be necessary to identify haplotypes linked to duplication alleles in these populations. Lastly, we were able to identify pathogenic or likely pathogenic SMN1 single nucleotide variants in 10 individuals, consistent with an overall carrier frequency of 0.15% in our cohort.

In summary, the NGS test reported herein is a sensitive and robust assay of SMN1 copy number and sequence variation that increases SMA carrier detection rates across all populations. In some embodiments, this approach can be integrated into existing NGS based carrier screening panels to improve SMA detection rates and reduce the overall cost of population carrier screening.

Further Methods

The Validation of NGS-Based Detection of g.27134T>G SNP—

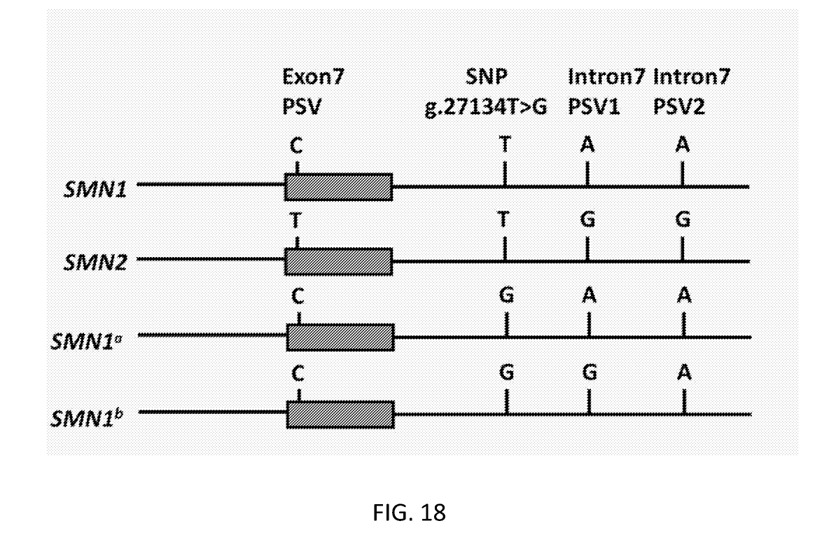

We developed a RFLP (Restriction fragment length polymorphism) analysis that can specifically detect the g.27134T>G SNP in the SMN1 locus (FIG. 17). Primers were designed to specifically amplify SMN1, but not SMN2, by utilizing the c.840C PSV at exon 7, as well as an additional mismatch nucleotide before the PSV. DNA with zero copy of SMN1 and two copies of SMN2 was included as a negative control to ensure no SMN2 copy is amplified nonspecifically. HpyCH4III cuts SMN1 PCR product only when SNP g.27134T>G is present. Next, we performed RFLP analysis and Sanger sequencing for 138 samples that are heterozygous, homozygous or negative for the g.27134T>G SNP on the SMN1 locus based on the capture NGS data (Table 9). All results were consistent among the capture NGS, RFLP and Sanger sequencing data. Additionally, we tested 12 samples that showed the g.27134T>G on the SMN2 locus by the capture NGS data. RFLP and Sanger sequencing results showed that these samples actually had the g.27134T>G SNP on the SMN1 locus. Careful examination of the NGS pileup data and Sanger sequencing results showed that the NGS misalignment was due to a novel haplotype where the intron 7 PSV1 is G instead of A in SMN1 (FIG. 18). In this situation, the g.27134T>G SNP was misaligned to the SMN2 locus by NGS alignment algorithm, while the SMN1 specific RFLP analysis was able to correctly identify the SNP in the SMN1 locus. A total of 493 consecutive samples by both NGS and RFLP analysis showed completely concordant results (Table 10).

TABLE 9
The g.27134T > G SNP positive and negative samples
detected by NGS were all confirmed by RFLP analysis and
Sanger sequencing.

| g.27134T > G SNP result | | | |
|---|---|---|---|
| from Capture NGS | Capture NGS | RFLP result | Sanger result |
| SNP heterozygous | 55 | 55 | 55 |
| SNP homozygous | 7 | 7 | 7 |
| SNP aligned to SMN2 | 12 | 12* | 12* |
| SNP negative | 77 | 77 | 77 |
| Total | 151 | 151 | 151 |

*All samples with SNP g.27134T > G aligned to SMN2 were subsequently confirmed by RFLP analysis and Sanger sequencing that they are actually located in SMN1.

TABLE 10
The g.27134T > G SNP positive and negative samples detected
by NGS capture data were all confirmed by RFLP analysis.

| g.27134T > G SNP result from Capture NGS | NGS | RFLP |
|---|---|---|
| SNP heterozygous | 58 | 58 |
| SNP homozygous | 3 | 3 |
| SNP aligned to SMN2 by NGS | 4 | 4* |
| SNP negative | 428 | 428 |
| Total | 493 | 493 |

*All samples with SNP g.27134T > G aligned to SMN2 were subsequently confirmed by RFLP analysis and Sanger sequencing that they are actually located in SMN1.

In summary, using an SMN1 specific RFLP analysis, we identified a novel haplotype that will cause g.27134T>G misalignment by NGS, in 0.8% of all samples. We also confirmed that all g.27134T>G SNP out of 493 samples were located at the SMN1 locus. An allelic change of g.27134T>G SNP on the SMN2 locus was not found.

Supplementary Methods and Procedures

SMN1 Gene Specific PCR and Sequencing—

Genomic regions containing exon 2-7 (5′ long fragment, 13 kb) and exon 7-8 (3′ short fragment, 1 kb) were amplified using long-range PCR reagents (TaKaRa LA Taq DNA Polymerase Hot-Start Version). Primers were designed to preferentially amplify SMN1 by utilizing the c.840C PSV at exon 7. For the short fragment, an additional mismatch base-pair before the PSV was also used to ensure SMN1 specificity.

Long fragment forward primer:
(SEQ ID NO: 39)
5′-GTGTGGATTAAGATGACTCTTGGTAC
Long fragment reverse primer:
(SEQ ID NO: 40)
5′-CACCTTCCTTCTTTTTGATTTTGTCTG
Short fragment forward primer:
(SEQ ID NO: 41)
5′-CTTCCTTTATTTTCCTTACAGGGTTCC
Short fragment reverse primer:
(SEQ ID NO: 42)
5′-TACAATGAACAGCCATGTCCAC

In a total volume of 25 μl, 1×LA PCR buffer, 2 ul of each primer (2.5 μM), 4 ul of dNTP (2.5 mM), 0.25 μl of TaKaRa LA Taq DNA Polymerase Hot-Start Version, and 1 ul of genomic DNA (50 ng/ul) were used. For the long fragment, denature 5 min at 95° C., followed by 38 cycles of 30 sec at 94° C., 45 sec at 66.5° C. and 15 min at 68° C., with a final extension for 5 min at 72° C. For the short fragment, denature 5 min at 95° C., then with 10 touchdown cycles of 45 sec at 94° C., 30 sec at 65-55° C. (each cycle with annealing temperature 1° C. lower than the previous cycle) and 60 sec at 72° C., followed by 20 regular cycles of 45 sec at 94° C., 30 sec at 55° C. and 60 sec at 72° C., with a final extension for 7 min at 72° C. The PCR products were then prepared for library construction for NGS.

Restriction Fragment Length Polymorphism Analysis for the g.27134T>G SNP—

RFLP (Restriction fragment length polymorphism) analysis for the silent carrier SNP (g.27134T>G) PCR was performed to amplify the SMN1 fragment containing the silent carrier SNP (g.27134T>G). Primers were designed to specifically amplify SMN1, but not SMN2, by utilizing the c.840C PSV at exon 7, as well as an additional mismatch basepair before the PSV. HpyCH4III will cut SMN1 PCR product only when SNP g.27134T>G is present. Forward primer was 5′-TGTAAAACGACGGCCAGTCTTCCTTTATTTTCCTTACAGGGTTGC (SEQ ID NO:43) and reverse primer was 5′-CAGGAAACAGCTATGACCAAGTCTGCTGGTCTGCCTACTAG (SEQ ID NO:44). In a total volume of 50 μl, 1×PCR buffer, 1 ul of each primer (10 μM), 4 ul of dNTP (2.5 mM), 0.25 μl of Platinum Taq polymerase (Invitrogen), 1.5 ul of MgCl₂ (50 mM), and 2 ul of genomic DNA (50 ng/ul) were used. Denature 2.5 min at 94° C., then with 10 touchdown cycles of 30 sec at 94° C., 30 sec at 65-50° C. (each cycle with annealing temperature 1.5° C. lower than the previous cycle) and 105 sec at 72° C., followed by 28 regular cycles of 30 sec at 94° C., 30 sec at 51° C. and 90 sec at 72° C., with a final extension for 5 min at 72° C. 10 ul PCR product was digested with 10U of HypCH4III (New England Biolabs, Cat# R0618) at 37° C. for 4 h and were resolved by 2% agarose gel electrophoresis.

### REFERENCES

All patents and publications mentioned in this specification are indicative of the level of those skilled in the art to which the invention pertains. All patents and publications herein are incorporated by reference to the same extent as if each individual publication was specifically and individually indicated to be incorporated by reference in their entirety.
1. Abuli A, Boada M, Rodriguez-Santiago B, et al. NGS-Based Assay for the Identification of Individuals Carrying Recessive Genetic Mutations in Reproductive Medicine. Hum Mutat. 2016; 37(6):516-523.
2. ADDENDUM: Technical standards and guidelines for spinal muscular atrophy testing. Genet Med. 2016; 18(7):752.
3. Arkblad E L, Darin N, Berg K, et al. Multiplex ligation-dependent probe amplification improves diagnostics in spinal muscular atrophy. Neuromuscul Disord. 2006; 16(12):830-838.
4. Cartegni L, Kraniner A R. Disruption of an SF2/ASF-dependent exonic splicing enhancer in SMN2 causes spinal muscular atrophy in the absence of SMN1. Nat Genet 2002; 30:377-384.
5. Contreras-Capetillo S N, Blanco H L, Cerda-Flores R M, et al. Frequency of SMN1 deletion carriers in a Mestizo population of central and northeastern Mexico: A pilot study. Exp Ther Med. 2015; 9(6):2053-2058.
6. Cusco I, Barcelo M J, Baiget M, Tizzano E F. Implementation of SMA carrier testing in genetic laboratories: comparison of two methods for quantifying the SMN1 gene. Hum Mutat. 2002; 20(6):452-459.
7. Cusco I, Barcelo M J, del Rio E, et al. Characterisation of SMN hybrid genes in Spanish SMA patients: de novo, homozygous and compound heterozygous cases. Hum Genet. 2001; 108(3):222-229.
8. Dubowitz V. Chaos in the classification of SMA: a possible resolution. Neuromuscul Disord. 1995; 5(1):3-5.
9. Emery A E, Hausmanowa-Petrusewicz I, Davie A M, Holloway S, Skinner R, Borkowska J. International collaborative study of the spinal muscular atrophies. Part 1. Analysis of clinical and laboratory data. J Neurol Sci. 1976; 29(1):83-94.
10. Feldkotter M, Schwarzer V, Wirth R, Wienker T F, Wirth B. Quantitative analyses of SMN1 and SMN2 based on real-time lightCycler PCR: fast and highly reliable carrier testing and prediction of severity of spinal muscular atrophy. Am J Hum Genet. 2002; 70(2):358-368.
11. Feng Y, Chen D, Wang G L, Zhang V W, Wong L J. Improved molecular diagnosis by the detection of exonic deletions with target gene capture and deep sequencing. Genet Med. 2015; 17(2):99-107.
12. Forreryd A, Johansson H, Albrekt A S, Lindstedt M. Evaluation of high throughput gene expression platforms using a genomic biomarker signature for prediction of skin sensitization. BMC Genomics. 2014; 15:379.
13. Hallam S, Nelson H, Greger V, et al. Validation for clinical use of, and initial clinical experience with, a novel approach to population-based carrier screening using high-throughput, next-generation DNA sequencing. J Mol Diagn. 2014; 16(2):180-189.
14. Haque I S, Lazarin G A, Kang H P, Evans E A, Goldberg J D, Wapner R J. Modeled Fetal Risk of Genetic Diseases Identified by Expanded Carrier Screening. JAMA. 2016; 316(7):734-742.
15. Hendrickson B C, Donohoe C, Akmaev V R, et al. Differences in SMN1 allele frequencies among ethnic groups within North America. J Med Genet. 2009; 46(9):641-644.
16. Kashima T, Manley J L. A negative element in SMN2 exon 7 inhibits splicing in spinal muscular atrophy. Nat Genet 2003; 34:460-463.
17. Larson J L, Silver A J, Chan D, Borroto C, Spurrier B, Silver L M. Validation of a high resolution NGS method for detecting spinal muscular atrophy carriers among phase 3 participants in the 1000 Genomes Project. BMC Med Genet. 2015; 16:100.
18. Lindsay S J, Khajavi M, Lupski J R, Hurles M E. A chromosomal rearrangement hotspot can be identified from population genetic variation and is coincident with a hotspot for allelic recombination. Am J Hum Genet. 2006; 79(5):890-902.
19. Liu C G, Calin G A, Meloon B, et al. An oligonucleotide microchip for genome-wide microRNA profiling in human and mouse tissues. Proc Natl Acad Sci USA. 2004; 101(26):9740-9744.
20. Lorson C L, Hahnen E, Androphy E J, Wirth B. A single nucleotide in the SMN gene regulates splicing and is responsible for spinal muscular atrophy. Proc Natl Acad Sci USA. 1999; 96(11):6307-6311.
21. Luo M, Liu L, Peter I, et al. An Ashkenazi Jewish SMN1 haplotype specific to duplication alleles improves pan-ethnic carrier screening for spinal muscular atrophy. Genet Med. 2014; 16(2):149-156.
22. MacDonald W K, Hamilton D, Kuhle S. SMA carrier testing: a meta-analysis of differences in test performance by ethnic group. Prenat Diagn. 2014; 34(12):1219-1226.
23. Mandelker D, Schmidt R J, Ankala A, et al. Navigating highly homologous genes in a molecular diagnostic setting: a resource for clinical next-generation sequencing. Genet Med. 2016.
24. McAndrew P E, Parsons D W, Simard L R, et al. Identification of proximal spinal muscular atrophy carriers and patients by analysis of SMNT and SMNC gene copy number. Am J Hum Genet. 1997; 60(6): 1411-1422.
25. Prior T W, Professional P, Guidelines C. Carrier screening for spinal muscular atrophy. Genet Med. 2008; 10(11):840-842.
26. Retterer K, Scuffins J, Schmidt D, et al. Assessing copy number from exome sequencing and exome array CGH based on CNV spectrum in a large clinical cohort. Genet Med. 2015; 17(8):623-629.
27. Schouten J P, McElgunn C J, Waaijer R, Zwijnenburg D, Diepvens F, Pals G. Relative quantification of 40 nucleic acid sequences by multiplex ligation-dependent probe amplification. Nucleic Acids Res. 2002; 30(12): e57.
28. Sheng-Yuan Z, Xiong F, Chen Y J, et al. Molecular characterization of SMN copy number derived from carrier screening and from core families with SMA in a Chinese population. Eur J Hum Genet. 2010; 18(9):978-984.
29. Sugarman E A, Nagan N, Zhu H, et al. Pan-ethnic carrier screening and prenatal diagnosis for spinal muscular atrophy: clinical laboratory analysis of >72,400 specimens. Eur J Hum Genet. 2012; 20(1):27-32.
30. Swoboda K J, Prior T W, Scott C B, et al. Natural history of denervation in SMA: relation to age, SMN2 copy number, and function. Ann Neurol. 2005; 57(5):704-712.
31. van der Steege G, Grootscholten P M, van der Vlies P, et al. PCR-based DNA test to confirm clinical diagnosis of autosomal recessive spinal muscular atrophy. Lancet. 1995; 345(8955):985-986.
32. Yang Y, Muzny D M, Reid J G, et al. Clinical whole-exome sequencing for the diagnosis of mendelian disorders. N Engl J Med. 2013; 369(16): 1502-1511.
33. Yang Y, Muzny D M, Xia F, et al. Molecular findings among patients referred for clinical whole-exome sequencing. JAMA. 2014; 312(18):1870-1879.

What is claimed is:

1. A method of determining gene copy number for an individual, comprising the step of identifying copy number of two nearly identical genes using sequencing data from next generation sequencing to distinguish at least one variance between the two genes.

2. The method of claim 1, wherein the identifying step comprises the determination of a mathematical relationship between a) the copy number ratio of the two genes, and b) the total copy number for both of the two genes in sum.

3. The method of claim 2, wherein the mathematical relationship is further defined as computing copy number for each gene by applying the copy number ratio to the total copy number.

4. The method of claim 1, 2, or 3, wherein the two genes are SMN1 and SMN2.

5. The method of any one of claims 1-4, wherein the gene copy number identifies carrier status for an individual.

6. The method of any one of claims 1-5, wherein the gene copy number is 0, 1, 2, 3, or more.

7. A method of assaying nucleic acid from a sample from an individual for a recessive allele for a genetic mutation associated with spinal muscular atrophy (SMA), comprising the step of generating a mathematical relationship between the total copy number of SMN1 and SMN2 and the copy number ratio of SMN1 to SMN2, wherein the total copy number and copy number ratio are determined using next generation sequencing data.

8. The method of claim 7, further comprising the step of determining that an individual is in need of assaying for the allele.

9. The method of claim 7 or 8, wherein the individual has a family history of SMA.

10. The method of claim 7 or 8, wherein the individual is pregnant.

11. The method of claim 7 or 8, wherein the individual is in need of family planning.

12. A method, comprising:
receiving sequenced sample data;
determining a copy number ratio between two nearly identical genes of the received sample data;
determining a total copy number of the two nearly identical genes of the received sample data; and
determining a final copy number for the two nearly identical genes for the received sample.

13. The method of claim 12, further comprising determining a patient outcome hypothesis based, at least in part, on the determined final copy number for the received sample corresponding to the patient.

14. The method of claim 13, wherein the step of determining the patient outcome hypothesis comprises determining that a patient is a carrier when the final copy number is not equal to two.

15. The method of claim 12, wherein the received sequenced sample data is received from next generation sequencing (NGS) and the sample data is aligned to hg19.

16. The method of claim 12, wherein the received sequenced sample data comprise a plurality of samples corresponding to a plurality of patients, and wherein a copy number ratio, a total copy number, and a final copy number is determined for each of the plurality of samples.

17. The method of claim 12, wherein the two nearly identical genes comprise the SMN1 and SMN2 genes.

18. The method of claim 12, wherein the step of determining the copy number ratio comprises:
reading a depth(rd) of PSVs for the received sample data;
calculating a copy number ratio for the received sample data for predetermined exons selected based on exons with expected differences; and
building a table of calculations for the calculated copy number ratios for a plurality of samples.

19. The method of claim 12, wherein the step of determining the total copy number comprises:
determining a total coverage of selected exons of the two nearly identical genes for each of a plurality of received samples;
determining a median or mean of each of the selected exons from samples having a ratio of the two nearly identical genes equal to approximately one;
normalizing the total coverage for the selected exons for each sample of the plurality of samples relative to all samples of the plurality of samples; and
determining the total copy number for each of the selected exons for each of the plurality of samples based, at least in part, on the normalized total coverage.

20. An apparatus comprising a processor and a memory, wherein the processor is coupled to the memory, and wherein the processor is configured to perform the steps recited in any of the preceding claims.

21. A computer program product, comprising: a non-transitory computer readable medium comprising code to perform steps comprising the steps recited in any of the preceding claims.
